SEQUENCE LISTING (24 sequences)

SEQ ID NO: 1 - DNA, 22 bp, Artificial Sequence
  Artificially synthesized primer sequence for RT-PCR
gttccaaaactgttcacttccc

SEQ ID NO: 2 - DNA, 23 bp, Artificial Sequence
  Artificially synthesized primer sequence for RT-PCR
ggtatggagactgatgaggacag

SEQ ID NO: 3 - DNA, 22 bp, Artificial Sequence
  Artificially synthesized primer sequence for RT-PCR
cttctgctggcctttctcctac

SEQ ID NO: 4 - DNA, 23 bp, Artificial Sequence
  Artificially synthesized primer sequence for RT-PCR
tgtggacgtttattaaggctctg

SEQ ID NO: 5 - DNA, 23 bp, Artificial Sequence
  Artificially synthesized primer sequence for RT-PCR
gaaccagctgtatttgttcaagg

SEQ ID NO: 6 - DNA, 23 bp, Artificial Sequence
  Artificially synthesized primer sequence for RT-PCR
aaaacaaaggtgagaagagaggg

SEQ ID NO: 7 - DNA, 23 bp, Artificial Sequence
  Artificially synthesized primer sequence for RT-PCR
tcctgaatgtgaagcagtatgtg

SEQ ID NO: 8 - DNA, 23 bp, Artificial Sequence
  Artificially synthesized primer sequence for RT-PCR
agccttgcattagttctcagcta

SEQ ID NO: 9 - DNA, 22 bp, Artificial Sequence
  Artificially synthesized primer sequence for RT-PCR
gtcccaagatgcatattttcct

SEQ ID NO: 10 - DNA, 23 bp, Artificial Sequence
  Artificially synthesized primer sequence for RT-PCR
ccgagcccattaatactgataga

SEQ ID NO: 11 - DNA, 23 bp, Artificial Sequence
  Artificially synthesized primer sequence for RT-PCR
actttctgacttaggccacaggt

SEQ ID NO: 12 - DNA, 23 bp, Artificial Sequence
  Artificially synthesized primer sequence for RT-PCR
acagagtgctcagttcttccgta

SEQ ID NO: 13 - DNA, 23 bp, Artificial Sequence
  Artificially synthesized primer sequence for RT-PCR
tctctgaccaagactgagaggac

SEQ ID NO: 14 - DNA, 23 bp, Artificial Sequence
  Artificially synthesized primer sequence for RT-PCR
gaggatacgaccgataggaactt

SEQ ID NO: 15 - DNA, 23 bp, Artificial Sequence
  Artificially synthesized primer sequence for RT-PCR
cagtcacaccaaggaagagaatg

SEQ ID NO: 16 - DNA, 23 bp, Artificial Sequence
  Artificially synthesized primer sequence for RT-PCR
cagtgaggattggatgaactagg

SEQ ID NO: 17 - DNA, 23 bp, Artificial Sequence
  Artificially synthesized primer sequence for RT-PCR
gtgtgattatcaaaagggagtgg

SEQ ID NO: 18 - DNA, 21 bp, Artificial Sequence
  Artificially synthesized primer sequence for RT-PCR
aatagtgcctatttaaggccg

SEQ ID NO: 19 - DNA, 22 bp, Artificial Sequence
  Artificially synthesized primer sequence for RT-PCR
tcctactttggccaagtttgtt

SEQ ID NO: 20 - DNA, 23 bp, Artificial Sequence
  Artificially synthesized primer sequence for RT-PCR
actaagctggtacatggaatgga

SEQ ID NO: 21 - DNA, 25 bp, Artificial Sequence
  Artificially synthesized primer sequence for RT-PCR
aaggagatggagtgtacaccttaaa

SEQ ID NO: 22 - DNA, 21 bp, Artificial Sequence
  Artificially synthesized primer sequence for RT-PCR
tgattgactcagcaatgcagg

SEQ ID NO: 23 - DNA, 23 bp, Artificial Sequence
  Artificially synthesized primer sequence for RT-PCR
catccacgaaactaccttcaact

SEQ ID NO: 24 - DNA, 23 bp, Artificial Sequence
  Artificially synthesized primer sequence for RT-PCR
tctccttagagagaagtggggtg

(19) United States
(12) Patent Application Publication
(10) Pub. No.: US 2007/0092519 A1
(43) Pub. Date: Apr. 26, 2007

(54) Method for diagnosing chronic myeloid leukemia

(75) Inventors: Yusuke Nakamura, Yokohama-shi (JP); Toyomasa Katagiri, Shinagawa-ku (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Onootherapy Science, Inc., Kawasaki-shi (JP); The University of Tokyo, Bunkyo-ku (JP)

(21) Appl. No.: 10/529,833

(22) PCT Filed: Aug. 12, 2003
(86) PCT No.: PCT/JP03/10256

§ 371 Date: Nov. 9, 2006

Related U.S. Application Data
(60) Provisional application No. 60/414,867, filed on Sep. 30, 2002.

(51) Int. Cl. A61K 39/395; C12Q 1/68; C12M 3/00; A61K 48/00

(52) U.S. Cl. 424/155.1; 514/44; 435/6; 435/287.2

(57) ABSTRACT

Objective methods for detecting and diagnosing Chronic myeloid leukemia (CML) are described herein. In one embodiment, the diagnostic method involves the determining a expression level of CML-associated gene that discriminate between CML and normal cell. The present invention further provides methods of screening for therapeutic agents useful in the treatment of CML, methods of treating CML and method of vaccinating a subject against CML.

### PRIORITY INFORMATION

This application claims priority to U.S. Provisional Application Ser. No. 60/414,867, filed Sep. 30, 2002.

### FIELD OF THE INVENTION

The invention relates to methods of diagnosing chronic myeloid leukemia.

### BACKGROUND OF THE INVENTION

Chronic myeloid leukemia (CML) is a clonal myeloproliferative disease characterized by Philadelphia (Ph) chromosome translocation (1). The resulting BCR-ABL fusion gene encodes a cytoplasmic protein that is constitutively activated for its tyrosine kinase activity. CML progresses through distinct clinical stages; the earliest stage, termed the chronic phase, is characterized by expansion of terminally differentiated neutrophils. The acute phase termed accelerated phase and blast crisis characterized by maturation arrest with excessive numbers of undifferentiated myeloid or lymphoid progenitor cells (2). Current therapies include allogenic stem-cell transplantation (SCT) and chemotherapies including interferon-α (IFN-α (3). IFN-α prolongs overall survival but has considerable adverse effects. SCT is the only curative treatment, but is associated with substantial morbidity and is limited to patients with suitable donors.

The development of the ABL-selective tyrosine kinase inhibitor STI571 (imanitib; Glivec; Novartis, Basel, Switzerland) is a significant advance in the management of CML (4, 5). STI571 frequently induces remarkable hematologic and cytogenetic responses in these clinical settings. However, recent clinical studies with STI571 in CML demonstrated that many patients at the advanced stage respond well but then relapse (6), (7). Resistance to STI571 because of enhanced expression or mutation of BCR-ABL gene has been found in CML patients (8), (9). Indeed, STI1571-induced hematologic responses occur less frequently and are less durable in CML patients at the blast crisis phase.

cDNA microarray technologies have enabled to obtain comprehensive profiles of gene expression in normal and malignant cells, and compare the gene expression in malignant and corresponding normal cells (Okabe et al., Cancer Res 61:2129-37 (2001); Kitahara et al., Cancer Res 61: 3544-9 (2001); Lin et al., Oncogene 21 :4120-8 (2002); Hasegawa et al., Cancer Res 62:7012-7 (2002)). This approach enables to disclose the complex nature of cancer cells, and helps to understand the mechanism of carcinogenesis. Identification of genes that are deregulated in tumors can lead to more precise and accurate diagnosis of individual cancers, and to develop novel therapeutic targets (Bienz and Clevers, Cell 103:311-20 (2000)). To disclose mechanisms underlying tumors from a genome-wide point of view, and discover target molecules for diagnosis and development of novel therapeutic drugs, the present inventors have been analyzing the expression profiles of tumor cells using a cDNA microarray of 23040 genes (Okabe et al., Cancer Res 61:2129-37 (2001); Kitahara et al., Cancer Res 61:3544-9 (2001); Lin et al., Oncogene 21:4120-8 (2002); Hasegawa et al., Cancer Res 62:7012-7 (2002)).

Studies designed to reveal mechanisms of carcinogenesis have already facilitated identification of molecular targets for anti-tumor agents. For example, inhibitors of farnexyltransferase (FTIs) which were originally developed to inhibit the growth-signaling pathway related to Ras, whose activation depends on posttranslational farnesylation, has been effective in treating Ras-dependent tumors in animal models (He et al., Cell 99:335-45 (1999)). Clinical trials on human using a combination or anti-cancer drugs and anti-HER2 monoclonal antibody, trastuzumab, have been conducted to antagonize the proto-oncogene receptor HER2/neu; and have been achieving improved clinical response and overall survival of breast-cancer patients (Lin et al., Cancer Res 61:6345-9 (2001)). A tyrosine kinase inhibitor, STI-571, which selectively inactivates bcr-abl fusion proteins, has been developed to treat chronic myelogenous leukemias wherein constitutive activation of bcr-abl tyrosine kinase plays a crucial role in the transformation of leukocytes. Agents of these kinds are designed to suppress oncogenic activity of specific gene products (Fujita et al., Cancer Res 61:7722-6 (2001)). Therefore, gene products commonly up-regulated in cancerous cells may serve as potential targets for developing novel anti-cancer agents.

It has been demonstrated that CD8+ cytotoxic T lymphocytes (CTLs) recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the discovered TAAs are now in the stage of clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products which had been demonstrated to be specifically overexpressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenberg et al., Nature Med 4: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomas, including colorectal cancer, are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and can der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-6 (1999); Oiso et al., Int J Cancer 81: 387-94(1999)).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in 51Cr-release assays (Kawano et al., Cance Res 60: 3550-8 (2000); Nishizaka et al., Cancer Res 60: 4830-7 (2000); Tanura et al., Jpn J Cancer Res 92: 762-7 (2001)). However, both of HLA-A24 and HLA-A0201 are one of the popular HLA alleles in Japanese, as well as Caucasian (Date et al., Tissue Antigens 47: 93-101 (1996); Kondo et al., J Immunol 155: 4307-12 (1995); Kubo et al., J Immunol 152: 3913-24 (1994); Imanishi et al., Proceeding of the eleventh International Histocompatibility Workshop and Conference Oxford University Press, Oxford, 1065 (1992); Williams et al., Tissue Antigen 49: 129 (1997)). Thus, antigenic peptides of carcinomas presented by these HLAs may be especially useful for the treatment of carcinomas among Japanese and Caucasian. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93: 4102-7 (1996)).

### SUMMARY OF THE INVENTION

The invention is based upon the discovery of a pattern of gene expression correlated with CML. The genes that are differentially expressed in CML are collectively referred to herein as “CML nucleic acids” or “CML polynucleotides” and the corresponding encoded polypeptides are referred to as “CML polypeptides” or “CML proteins.”

Accordingly, the invention features a method of diagnosing or determining a predisposition to CML in a subject by determining an expression level of a CML-associated gene in a patient derived biological sample, such as peripheral blood sample or myeloid cells sample. By CML associated gene is meant a gene that is characterized by an expression level which differs in a cell containing a Philadelphia (Ph) chromosome translocation or in a cell obtained from an individual with a family history of CML or an individual exhibiting clinical symptoms of CML, compared to a normal peripheral blood cell. A CML-associated gene is one or more of CML 1-296. An alteration, e.g., increase or decrease of the level of expression of the gene compared to a normal control level of the gene indicates that the subject suffers from or is at risk of developing CML.

By normal control level is meant a level of gene expression detected in a normal, healthy individual or in a population of individuals known not to be suffering from CML. A control level is a single expression pattern derived from a single reference population or from a plurality of expression patterns. For example, the control level can be a database of expression patterns from previously tested cells. A normal individual is one with no clinical symptoms of CML and no family history of CML.

An increase in the level of CML 1-190 detected in a test sample compared to a normal control level indicates the subject (from which the sample was obtained) suffers from or is at risk of developing CML. In contrast, a decrease in the level of CML 191-296 detected in a test sample compared to a normal control level indicates said subject suffers from or is at risk of developing CML.

Alternatively, expression of a panel of CML-associated genes in the sample is compared to a CML control level of the same panel of genes. By CML control level is meant the expression profile of the CML-associated genes found in a population suffering from CML.

Gene expression is increased or decreased 10%, 25%, 50% compared to the control level. Alternately, gene expression is increased or decreased 1, 2, 5 or more fold compared to the control level. Expression is determined by detecting hybridization, e.g., on an array, of a CML-associated gene probe to a gene transcript of the patient-derived cell sample.

The patient-derived cell sample is any cell from a test subject, e.g., a patient known to or suspected of having CML. For example, the sample contains a mixture of mononuclear cells from peripheral blood.

The invention also provides a CML reference expression profile of a gene expression level of two or more of CML 1-296. Alternatively, the invention provides a CML reference expression profile of the levels of expression of two or more of CML 1-190 or CML 191-296.

The invention further provides methods of identifying an agent that inhibits or enhances the expression or activity of a CML-associated gene, e.g. CML 1-296 by contacting a test cell expressing a CML associated gene with a test agent and determining the expression level of the CML associated gene. The test cell is a mononuclear cell such as a mononuclear cell from peripheral blood of CML patient. A decrease of the level compared to a normal control level of the gene indicates that the test agent is an inhibitor of the CML-associated gene and reduces a symptom of CML, e.g., CML 1-190. Alternatively, an increase of the level or activity compared to a normal control level or activity of the gene indicates that said test agent is an enhancer of expression or function of the CML-associated gene and reduces a symptom of CML, e.g., CML 191-296.

The invention also provides a kit with a detection reagent which binds to two or more CML nucleic acid sequences or which binds to a gene product encoded by the nucleic acid sequences. Also provided is an array of nucleic acids that binds to two or more CML nucleic acids.

Therapeutic methods include a method of treating or preventing CML in a subject by administering to the subject an antisense composition. The antisense composition reduces the expression of a specific target gene, e.g., the antisense composition contains a nucleotide, which is complementary to a sequence selected from the group consisting of CML 1-190. Another method includes the steps of administering to a subject an short interfering RNA (siRNA) composition. The siRNA composition reduces the expression of a nucleic acid selected from the group consisting of CML 1-190. In yet another method, treatment or prevention of CML in a subject is carried out by administering to a subject a ribozyme composition. The nucleic acid-specific ribozyme composition reduces the expression of a nucleic acid selected from the group consisting of CML 1-190. Other therapeutic methods include those in which a subject is administered a compound that increases the expression of CML 191-296 or activity of a polypeptide encoded by CML 191-296. Furthermore, CML can be treated by administering a protein encoded by CML 191-296. The protein may be directly administered to the patient or, alternatively, may be expressed in vivo subsequent to being introduced into the patient, for example, by administering an expression vector or host cell carrying the down-regulated marker gene of interest. Suitable mechanisms for in vivo expression of a gene of interest are known in the art.

The invention also includes vaccines and vaccination methods. For example, a method of treating or preventing CML in a subject is carried out by administering to the subject a vaccine containing a polypeptide encoded by a nucleic acid selected from the group consisting of CML 1-190 or an immunologically active fragment such a polypeptide. An immunologically active fragment is a polypeptide that is shorter in length than the full-length naturally-occurring protein and which induces an immune response. For example, an immunologically active fragment at least 8 residues in length and stimulates an immune cell such as a T cell or a B cell. Immune cell stimulation is measured by detecting cell proliferation, elaboration of cytokines (e.g., IL-2), or production of an antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms such as expansion of terminally-differentiated neutrophils. Other features and advantages of the invention will be apparent from the following detailed description, and from the claims.

### BRIEF DESCRIPTION OF THE FIGURES

FIG. 1 is a photograph of a DNA agarose gel showing expression of representative 11 genes and β-actin examined by semi-quantitative RT-PCR using cDNA prepared from amplified RNA. The first lane shows the expression level of each gene in a normal individual. The remaining eight lanes each show the expression level of the genes in a different CML patient. Gene symbols are noted for genes whose function was known or inferred and Accession No. Noted for ESTs.

### DETAILED DESCRIPTION

The present invention is based in part on the discovery of changes in expression patterns of multiple nucleic acid sequences in mononuclear cells from peripheral blood of patients with Chronic myeloid leukemia (CML). The differences in gene expression were identified by using a comprehensive cDNA microarray system.

Using a cDNA microarray containing 23,040 genes, comprehensive gene-expression profiles were obtained of 27 CMLs. Two hundred ninety-six genes were found to be differentially expressed in mononuclear cells from peripheral blood. Results show that certain genes are expressed at low or high levels in CMLs. Selection was made of candidate molecular markers with the potential of detecting CML-related proteins in blood, serum, or sputum of patients, and discovered some potential targets for development of signal-suppressing strategies in CML.

The differentially expressed genes identified herein are used for diagnostic purposes as markers of CML as gene targets, the expression of which is altered to treat or alleviate a symptom of CML.

The genes whose expression levels are modulated (i.e., increased or decreased) in CML patients are summarized in Tables 3-4 and are collectively referred to herein as “CML-associated genes”, “CML nucleic acids” or “CML polynucleotides” and the corresponding encoded polypeptides are referred to as “CML polypeptides”, or “CML proteins.” Unless indicated otherwise, “CML” is meant to refer to any of the sequences disclosed herein. (e.g., CML 1-296). The genes that have been previously described are presented along with a database accession number.

By measuring expression of the various genes in a sample of cells, CML is diagnosed. Similarly, by measuring the expression of these genes in response to various agents can identify agents for treating CML.

The invention involves determining (e.g., measuring) the expression of at least one, and up to all the CML sequences listed in Tables 3-4. Using sequence information provided by the GeneBank™ database entries for the known sequences the CML associated genes are detected and measured using techniques well known to one of ordinary skill in the art. For example, sequences within the sequence database entries corresponding to CML sequences, are used to construct probes for detecting CML RNA sequences in, e.g., northern blot hybridization analyses. Probes include at least 10, 20, 50, 100, 200 nucleotides of a reference sequence. As another example, the sequences can be used to construct primers for specifically amplifying the CML sequences in, e.g., amplification-based detection methods such as reverse-transcription based polymerase chain reaction.

Expression level of one or more of the CML sequences in the test cell population, e.g., a patient derived cell sample is then compared to expression levels of the same sequences in a reference population. The reference cell population includes one or more cells for which the compared parameter is known, i.e., CML cells or non-CML cells.

Whether or not a pattern of gene expression in the test cell population compared to the reference cell population indicates CML or a predisposition thereto depends upon on the composition of the reference cell population. For example, if the reference cell population is composed of non-CML cells, a similar gene expression pattern in the test cell population and reference cell population indicates the test cell population is non-CML. Conversely, if the reference cell population is made up of CML cells, a similar gene expression profile between the test cell population and the reference cell population indicates that the test cell population includes CML cells.

A level of expression of a CML marker gene in a test cell population is considered altered in levels of expression if its expression level varies from the reference cell population by more than 1.0, 1.5, 2.0, 5.0, 10.0 or more fold from the expression level of the corresponding CML sequence in the reference cell population.

Differential gene expression between a test cell population and a reference cell population is normalized to a control nucleic acid, e.g. a housekeeping gene. For example, a control nucleic acid is one which is known not to differ depending on the CML or non-CML state of the cell. Expression levels of the control nucleic acid in the test and reference nucleic acid can be used to normalize signal levels in the compared populations. Control genes include β-actin, glyceraldehyde 3-phosphate dehydrogenase or ribosomal protein P1.

The test cell population is compared to multiple reference cell populations. Each of the multiple reference populations may differ in the known parameter. Thus, a test cell population may be compared to a second reference cell population known to contain, e.g., CML cells, as well as a second reference population known to contain, e.g., non-CML cells (normal cells). The test cell is included in a tissue type or cell sample from a subject known to contain, or to be suspected of containing, CML cells.

The test cell is obtained from a bodily tissue or a bodily fluid, e.g., biological fluid (such as blood or sputum). For example, the test cell is purified from a tissue. Preferably, the test cell population comprises a mononuclear cell.

Cells in the reference cell population are derived from a tissue type similar to test cell. Optionally, the reference cell population is a cell line, e.g. a CML cell line (positive control) or a normal non-CML cell line (negative control). Alternatively, the control cell population is derived from a database of molecular information derived from cells for which the assayed parameter or condition is known.

The subject is preferably a mammal. The mammal can be, e.g., a human, non-human primate, mouse, rat, dog, cat, horse, or cow.

Expression of the genes disclosed herein is determined at the RNA level using any method known in the art. For example, Northern hybridization analysis using probes which specifically recognize one or more of these sequences can be used to determine gene expression. Alternatively, expression is measured using reverse-transcription-based PCR assays, e.g., using primers specific for the differentially expressed sequences. Expression is also determined at the protein level, i.e., by measuring the levels of polypeptides encoded by the gene products described herein, or biological activity thereof. Such methods are well known in the art and include, e.g., immunoassays based on antibodies to proteins encoded by the genes. The biological activity of the proteins encoded by the genes are also well known.

Diagnosing CML

CML is diagnosed by measuring the level of expression of one or more CML nucleic acid sequences from a test population of cells, (i.e., a patient derived biological sample). Preferably, the test cell population contains a mononuclear cell, e.g., a cell obtained from peripheral blood. Other biological samples can be used for measuring the protein level. For example, the protein level in the blood, or serum, derived from subject to be diagnosed can be measured by immunoassay or biological assay.

Expression of one or more of CML-associated genes, e.g., CML 1-296 is determined in the test cell or biological sample and compared to the expression of the normal control level. A normal control level is an expression profile of a CML-associated gene typically found in a population known not to be suffering from CML. An increase or a decrease of the level of expression in the patient derived cell sample of the CML-associated genes indicates that the subject is suffering from or is at risk of developing CML. For example, an increase in expression of CML 1-190 in the test population compared to the normal control level indicates that the subject is suffering from or is at risk of developing CML. Conversely, a decrease in expression of CML 191-296 in the test population compared to the normal control level indicates that the subject is suffering from or is at risk of developing CML.

When one or more of the CML-associated genes are altered in the test population compared to the normal control level indicates that the subject suffers from or is at risk of developing CML. For example, at least 1%, 5%, 25%, 50%, 60%, 80%, 90% or more of the panel of CML-associated genes (CML 1-190, CML 191-296, or CML 1-296) are altered.

Identifying Agents that Inhibit or Enhance CML-Associated Gene Expression

An agent that inhibits the expression or activity of a CML-associated gene is identified by contacting a test cell population expressing a CML associated up-regulated gene with a test agent and determining the expression level of the CML associated gene. A decrease in expression in the presence of the agent compared to the normal control level (or compared to the level in the absence of the test agent) indicates the agent is an inhibitor of a CML associated up-regulated gene and useful to inhibit CML.

Alternatively, an agent that enhances the expression or activity of a CML down-regulated associated gene is identified by contacting a test cell population expressing an CML associated gene with a test agent and determining the expression level or activity of the CML associated down-regulated gene. An increase of expression or activity compared to a normal control level or activity of the CML-associated gene indicates that the test agent augments expression or activity of the down-regulated CML associated gene.

The test cell population is any cell expressing the CML-associated genes. For example, the test cell population contains a mononuclear cell, such a cell is isolated from peripheral blood. Alternatively, the test cell is a cell, which has been transfected with a CML associated gene or which has been transfected with a regulatory sequence (e.g. promoter sequence) from a CML-associated gene operably linked to a reporter gene.

Assessing Efficacy of Treatment of CML in a Subject

The differentially expressed CML sequences identified herein also allow for the course of treatment of CML to be monitored. In this method, a test cell population is provided from a subject undergoing treatment for CML. If desired, test cell populations are obtained from the subject at various time points before, during, or after treatment. Expression of one or more of the CML sequences, in the cell population is then determined and compared to a reference cell population which includes cells whose CML state is known. The reference cells have not been exposed to the treatment.

If the reference cell population contains no CML cells, a similarity in expression between CML sequences in the test cell population and the reference cell population indicates that the treatment is efficacious. However, a difference in expression between CML sequences in the test population and a normal control reference cell population indicates a less favorable clinical outcome or prognosis.

By “efficacious” is meant that the treatment leads to a reduction in expression of a pathologically upregulated gene, increase in expression of a pathologically down-regulated gene or a decrease leukemic stem cells and their dividing progeny (i.e., granulocytic, erythroid, and megakaryocytic precursors) in a subject. When treatment is applied prophylactically, “efficacious” means that the treatment retards or prevents a symptom of clinical CML. For example, the treatment inhibits a symptom of chronic, acute, or accelerated phase. Assessment of CML is made using standard clinical protocols.

Efficaciousness is determined in association with any known method for diagnosing or treating CML. CML is diagnosed for example, by identifying symptomatic anomalies, e.g., anemia, hypermetabolism, easy fatigability, weakness, weight loss, and anorexia. Other characteristics of CML include splenomegaly, thrombocytosis and an almost total lack of alkaline phosphatase in granulocytes. Patients also exhibit marked elevation of the leukocyte count with the circulating cells being predominantly neutrophils and metamyelocytes, but basophils and eosinophils may also be prominent. Furthermore, the Ph¹ (Philadelphia) chromosome is present in the dividing progeny of multipotent myeloid stem cells (i.e., granulocytic, erythroid, and megakaryocytic precursors) and lymphoid cells (i.e., B cells) of approximately 90% of patients with CML.

Selecting a Therapeutic Agent for Treating CML that is Appropriate for a Particular Individual

Differences in the genetic makeup of individuals can result in differences in their relative abilities to metabolize various drugs. An agent that is metabolized in a subject to act as an anti-CML agent can manifest itself by inducing a change in gene expression pattern in the subject's cells from that characteristic of a CML state to a gene expression pattern characteristic of a non-CML state. Accordingly, the differentially expressed CML sequences disclosed herein allow for a putative therapeutic or prophylactic inhibitor of CML to be tested in a test cell population from a selected subject in order to determine if the agent is a suitable anti-CML agent in the subject.

To identify an inhibitor or enhancer of CML, that is appropriate for a specific subject, a test cell population from the subject is exposed to a therapeutic agent, and the expression of one or more of CML 1-296 sequences is determined.

The test cell population contains an CML cell expressing an CML associated gene. Preferably, the test cell is a mononuclear cell. For example a test cell population is incubated in the presence of a candidate agent and the pattern of gene expression of the test sample is measured and compared to one or more reference profiles, e.g., a CML reference expression profile or an non-CML reference expression profile.

A decrease in expression of one or more of the sequences CML 1-190 or an increase in expression of one or more of the sequences CML 191-296 in a test cell population relative to a reference cell population containing CML is indicative that the agent is therapeutic.

The test agent can be any compound or composition. For example, the test agents are agents that regulate growth and differentiation of hematopoietic precursors.

Screening Assays for Identifying Therapeutic Agents

The differentially expressed sequences disclosed herein can also be used to identify candidate therapeutic agents for treating a CML. The method is based on screening a candidate therapeutic agent to determine if it converts an expression profile of CML 1-296 sequences characteristic of a CML state to a pattern indicative of a non-CML state.

In the method, a cell is exposed to a test agent or a combination of test agents (sequentially or consequentially) and the expression of one or more CML 1-296 sequences in the cell is measured. The expression profile of the CML sequences in the test population is compared to expression level of the CML sequences in a reference cell population that is not exposed to the test agent.

An agent effective in stimulating expression of underexpressed genes, or in suppressing expression of overexpressed genes is deemed to lead to a clinical benefit such compounds are further tested for the ability to prevent an increased myeloid stem cell mass or to prevent maturation of leukemic stem cells (i.e., pluripotent hematopoietic stem cells), in animals or test subjects.

In a further embodiment, the present invention provides methods for screening candidate agents which are potential targets in the treatment of CML. As discussed in detail above, by controlling the expression levels or activities of marker genes, one can control the onset and progression of CML. Thus, candidate agents, which are potential targets in the treatment of CML, can be identified through screenings that use the expression levels and activities of marker genes as indices. In the context of the present invention, such screening may comprise, for example, the following steps:
a) contacting a test compound with a polypeptide encoded by CML 1-296;
b) detecting the binding activity between the polypeptide and the test compound; and
c) selecting a compound that binds to the polypeptide

Alternatively, the screening method of the present invention may comprise the following steps:
a) contacting a candidate compound with a cell expressing one or more marker genes, wherein the one or more marker genes is selected from the group consisting of CML 1-296; and
b) selecting a compound that reduces the expression level of one or more marker genes selected from the group consisting of CML 1-190, or elevates the expression level of one or more marker genes selected from the group consisting of CML 191-296.
   Cells expressing a marker gene include, for example, cell lines established from CML; such cells can be used for the above screening of the present invention.

Alternatively, the screening method of the present invention may comprise the following steps:
a) contacting a test compound with a polypeptide encoded by selected from the group consisting of CML 1-296;
b) detecting the biological activity of the polypeptide of step (a); and
c) selecting a compound that suppresses the biological activity of the polypeptide encoded by CML 1-190 in comparison with the biological activity detected in the absence of the test compound, or enhances the biological activity of the polypeptide encoded by CML 191-296 in comparison with the biological activity detected in the absence of the test compound.

A protein required for the screening can be obtained as a recombinant protein using the nucleotide sequence of the marker gene. Based on the information of the marker gene, one skilled in the art can select any biological activity of the protein as an index for screening and a measurement method based on the selected biological activity.

Alternatively, the screening method of the present invention may comprise the following steps:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of CML 1-296
b) measuring the activity of said reporter gene; and
c) selecting a compound that reduces the expression level of said reporter gene when said marker gene is an up-regulated marker gene selected from the group consisting of CML 1-190 or that enhances the expression level of said reporter gene when said marker gene is a down-regulated marker gene selected from the group consisting of CML 191-296, as compared to a control.

Suitable reporter genes and host cells are well known in the art. The reporter construct required for the screening can be prepared by using the transcriptional regulatory region of a marker gene. When the transcriptional regulatory region of a marker gene has been known to those skilled in the art, a reporter construct can be prepared by using the previous sequence information. When the transcriptional regulatory region of a marker gene remains unidentified, a nucleotide segment containing the transcriptional regulatory region can be isolated from a genome library based on the nucleotide sequence information of the marker gene.

The compound isolated by the screening is a candidate for drugs that inhibit the activity of the protein encoded by marker genes and can be applied to the treatment or prevention of CML.

Moreover, compound in which a part of the structure of the compound inhibiting the activity of proteins encoded by marker genes is converted by addition, deletion and/or replacement are also included in the compounds obtainable by the screening method of the present invention.

When administrating the compound isolated by the method of the invention as a pharmaceutical for humans and other mammals, such as mice, rats, guinea-pigs, rabbits, chicken, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; and flavoring agents such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water used for injections.

Physiological saline, glucose, and other isotonic liquids including adjuvants, such as D-sorbitol, D-mannose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80 (TM) and HCO-50.

Sesame oil or Soy-bean oil can be used as a oleaginous liquid and may be used in conjunction with benzyl benzoate or benzyl alcohol as a solubilizer and may be formulated with a buffer, such as phosphate buffer and sodium acetate buffer, a pain-killer, such as procaine hydrochloride; a stabilizer, such as benzyl alcohol and phenol; and an anti-oxidant. The prepared injection may be filled into a suitable ampule.

Methods well known to one skilled in the art may be used to administer the pharmaceutical composition of the present invention to patients, for example as intraarterial, intravenous, or percutaneous injections and also as intranasal, transbronchial, intramuscular or oral administrations. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient but one skilled in the art can suitably select them.

For example, although the dose of a compound that binds to the protein of the present invention and regulates its activity depends on the symptoms, the dose is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kgs of body-weight.

Assessing the Prognosis of a Subject with CML

Also provided is a method of assessing the prognosis of a subject with CML by comparing the expression of one or more CML sequences in a test cell population to the expression of the sequences in a reference cell population derived from patients over a spectrum of disease stages. By comparing gene expression of one or more CML sequences in the test cell population and the reference cell population(s), or by comparing the pattern of gene expression over time in test cell populations derived from the subject, the prognosis of the subject can be assessed.

A decrease in expression of one or more of the sequences CML 191-296 compared to a normal control or an increase of expression of one or more of the sequences CML 1-190 compared to a normal control indicates less favorable prognosis. An increase in expression of one or more of the sequences CML 191-296 indicates a more favorable prognosis, and a decrease in expression of sequences CML 1-190 indicates a more favorable prognosis for the subject.

Kits

The invention also includes a CML-detection reagent, e.g., a nucleic acid that specifically binds to or identifies one or more CML nucleic acids such as oligonucleotide sequences, which are complementary to a portion of a CML nucleic acid or antibodies which bind to proteins encoded by a CML nucleic acid. The reagents are packaged together in the form of a kit. The reagents are packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay are included in the kit. The assay format of the kit is a Northern hybridization or a sandwich ELISA known in the art.

For example, CML detection reagent, is immobilized on a solid matrix such as a porous strip to form at least one CML detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of CML present in the sample. The detection sites may be configured in any suitably detectable shape and are typically in the shape of a bar or dot spanning the width of a teststrip.

Alternatively, the kit contains a nucleic acid substrate array comprising one or more nucleic acid sequences. The nucleic acids on the array specifically identify one or more nucleic acid sequences represented by CML 1-296. The expression of 2, 3, 4, 5, 6, 7, 8, 9, 10, 15, 20, 25, 40 or 50 or more of the sequences represented by CML 1-296 are identified by virtue of the level of binding to an array test strip or chip. The substrate array can be on, e.g., a solid substrate, e.g., a “chip” as described in U.S. Pat. No. 5,744,305.

Arrays and Pluralities

The invention also includes a nucleic acid substrate array comprising one or more nucleic acid sequences. The nucleic acids on the array specifically correspond to one or more nucleic acid sequences represented by CML 1-296. The level of expression of 2, 3, 4, 5, 6, 7, 8, 9, 10, 15, 20, 25, 40 or 50 or more of the sequences represented by CML 1-296 are identified by detecting nucleic acid binding to the array.

The invention also includes an isolated plurality (i.e., a mixture of two or more nucleic acids) of nucleic acid sequences. The nucleic acid sequences are in a liquid phase or a solid phase, e.g., immobilized on a solid support such as a nitrocellulose membrane. The plurality includes one or more of the nucleic acid sequences represented by CML 1-296. In various embodiments, the plurality includes 2, 3, 4, 5, 6, 7, 8, 9, 10, 15, 20, 25, 40 or 50 or more of the sequences represented by CML 1-296.

Methods of Inhibiting CML

The invention provides a method for treating or alleviating a symptom of CML in a subject by decreasing expression or activity of CML 1-190 or increasing expression or activity of CML 191-296. Therapeutic compounds are administered prophylactically or therapeutically to a subject suffering from or at risk of (or susceptible to) developing CML. Such subjects are identified using standard clinical methods or by detecting an aberrant level of expression or activity of (e.g., CML 1-296). Therapeutic agents include inhibitors of cell cycle regulation, cell proliferation, and protein kinase activity. Preferably, the inhibitor of kinase activity is not STI571. Alternatively, STI571 is administered together with one or more of the inhibitors of CML 1-296.

The therapeutic method includes increasing the expression, or function, or both of one or more gene products of genes whose expression is decreased (“underexpressed genes”) in a CML cell relative to normal cells of the same cell type from which the CML cells are derived. In these methods, the subject is treated with an effective amount of a compound, which increases the amount of one of more of the underexpressed genes in the subject. Administration can be systemic or local. Therapeutic compounds include a polypeptide product of an underexpressed gene, or a biologically active fragment thereof, a nucleic acid encoding an underexpressed gene and having expression control elements permitting expression in the CML cells; for example an agent which increases the level of expression of such gene endogenous to the CML cells (i.e., which up-regulates expression of the underexpressed gene or genes). Administration of such compounds counter the effects of aberrantly-under expressed of the gene or genes in the subject's hematopoietic cells and improves the clinical condition of the subject.

The method also includes decreasing the expression, or function, or both, of one or more gene products of genes whose expression is aberrantly increased (“overexpressed gene”) in hematopoietic cells including hematopoietic stem cells. Expression is inhibited in any of several ways known in the art. For example, expression is inhibited by administering to the subject a nucleic acid that inhibits, or antagonizes, the expression of the overexpressed gene or genes, e.g., an antisense oligonucleotide or small interfering RNA which disrupts expression of the overexpressed gene or genes.

As noted above, antisense nucleic acids corresponding to the nucleotide sequence of CML 1-190 can be used to reduce the expression level of the CML 1-190. Antisense nucleic acids corresponding to CML 1-190 that are up-regulated in CML are useful for the treatment of CML. Specifically, the antisense nucleic acids of the present invention may act by binding to the CML 1-190 or mRNAs corresponding thereto, thereby inhibiting the transcription or translation of the genes, promoting the degradation of the mRNAs, and/or inhibiting the expression of proteins encoded by the CML 1-190, finally inhibiting the function of the proteins . The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at 80% or higher, more preferably 90% or higher, even more preferably 95% or higher over a span of at least 15 continuous nucleotides. Algorithms known in the art can be used to determine the homology.

The antisense nucleic acid derivatives of the present invention act on cells producing the proteins encoded by marker genes by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid derivative of the present invention can be made into an external preparation, such as a liniment or a poultice, by mixing with a suitable base material which is inactive against the derivative.

Also, as needed, the derivatives can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids derivative is given to the patient by directly applying onto the ailing site or by injecting into a blood vessel so that it will reach the site of ailment. An antisense-mounting medium can also be used to increase durability and membrane-permeability. Examples are, liposomes, poly-L-lysine, lipids, cholesterol, lipofectin or derivatives of these.

The dosage of the antisense nucleic acid derivative of the present invention can be adjusted suitably according to the patient's condition and used in desired amounts. For example, a dose range of 0.1 to 100 mg/kg, preferably 0.1 to 50 mg/kg can be administered.

The antisense nucleic acids of the invention inhibit the expression of the protein of the invention and is thereby useful for suppressing the biological activity of a protein of the invention. Also, expression-inhibitors, comprising the antisense nucleic acids of the invention, are useful since they can inhibit the biological activity of a protein of the invention.

The antisense nucleic acids of present invention include modified oligonucleotides. For example, thioated nucleotides may be used to confer nuclease resistance to an oligonucleotide.

Also, an siRNA against marker gene can be used to reduce the expression level of the marker gene. By the term “siRNA” is meant a double stranded RNA molecule which prevents translation of a target mRNA. Standard techniques of introducing siRNA into the cell are used, including those in which DNA is a template from which RNA is transcribed. In the context of the present invention, the siRNA comprises a sense nucleic acid sequence and an anti-sense nucleic acid sequence against an upregulated marker gene, such as CML 1-190. The siRNA is constructed such that a single transcript has both the sense and complementary antisense sequences from the target gene, e.g., a hairpin.

The method is used to alter the expression in a cell of an upregulated, e.g., as a result of malignant transformation of the cells. Binding of the siRNA to a transcript corresponding to one of the CML 1-190 in the target cell results in a reduction in the protein production by the cell. The length of the oligonucleotide is at least 10 nucleotides and may be as long as the naturally-occurring the transcript. Preferably, the oligonucleotide is 19-25 nucleotides in length. Most preferably, the oligonucleotide is less than 75, 50, 25 nucleotides in length.

The nucleotide sequence of the siRNAs were designed using an siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:
1. Beginning with the AUG start codon of the object transcript, scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.
2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/
3. Select qualifying target sequences for synthesis. At Ambion, preferably several target sequences can be selected along the length of the gene for evaluation.

The antisense oligonucleotide or siRNA of the invention inhibit the expression of the polypeptide of the invention and is thereby useful for suppressing the biological activity of the polypeptide of the invention. Also, expression-inhibitors, comprising the antisense oligonucleotide or siRNA of the invention, are useful in the point that they can inhibit the biological activity of the polypeptide of the invention. Therefore, a composition comprising the antisense oligonucleotide or siRNA of the present invention are useful in treating a CML.

Alternatively, function of one or more gene products of the overexpressed genes is inhibited by administering a compound that binds to or otherwise inhibits the function of the gene products. For example, the compound is an antibody which binds to the overexpressed gene product or gene products.

The present invention refers to the use of antibodies, particularly antibodies against a protein encoded by an up-regulated marker gene, or a fragment of the antibody. As used herein, the term “antibody” refers to an immunoglobulin molecule having a specific structure, that interacts (i.e., binds) only with the antigen that was used for synthesizing the antibody (i.e., the up-regulated marker gene product) or with an antigen closely related to it Furthermore, an antibody may be a fragment of an antibody or a modified antibody, so long as it binds to one or more of the proteins encoded by the marker genes. For instance, the antibody fragment may be Fab, F(ab′)2, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horowitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. These modification methods are conventional in the field.

Alternatively, an antibody may be obtained as a chimeric antibody, between a variable region derived from a nonhuman antibody and a constant region derived from a human antibody, or as a humanized antibody, comprising the complementarity determining region (CDR) derived from a nonhuman antibody, the frame work region (FR) derived from a human antibody, and the constant region. Such antibodies can be prepared by using known technologies.

Cancer therapies directed at specific molecular alterations that occur in cancer cells have been validated through clinical development and regulatory approval of anti-cancer drugs such as trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F, Tortora G. A novel approach in the treatment of cancer: targeting the epidermal growth factor receptor. Clin Cancer Res. 2001 October; 7(10):2958-70. Review.; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J Med. 2001 Mar. 15; 344(11):783-92.; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. 2003 Jan. 15; 101(2):420-424.; Fang G, Kim C N, Perkins C L, Ramadevi N, Winton E, Wittmann S and Bhalla K N. (2000). Blood, 96, 2246-2253.). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56.; Klejman A, Rushen L, Morrione A, Slupianek A and Skorski T. (2002). Oncogene, 21, 5868-5876.). Therefore, future cancer treatments will probably involve combining conventional drugs with target-specific agents aimed at different characteristics of tumor cells such as angiogenesis and invasiveness.

These modulatory methods are performed ex vivo or in vitro (e.g., by culturing the cell with the agent) or, alternatively, in vivo (e.g., by administering the agent to a subject). The method involves administering a protein or combination of proteins or a nucleic acid molecule or combination of nucleic acid molecules as therapy to counteract aberrant expression or activity of the differentially expressed genes.

Diseases and disorders that are characterized by increased (relative to a subject not suffering from the disease or disorder) levels or biological activity of the genes may be treated with therapeutics that antagonize (i.e., reduce or inhibit) activity of the overexpressed gene or genes. Therapeutics that antagonize activity are administered therapeutically or prophylactically.

Therapeutics that may be utilized include, e.g., (i) a polypeptide, or analogs, derivatives, fragments or homologs thereof of the overexpressed or underexpressed sequence or sequences; (ii) antibodies to the overexpressed or underexpressed sequence or sequences; (iii) nucleic acids encoding the over or underexpressed sequence or sequences; (iv) antisense nucleic acids or nucleic acids that are “dysfunctional” (i.e., due to a heterologous insertion within the coding sequences of coding sequences of one or more overexpressed or underexpressed sequences); (v) small interfering RNA (siRNA); or (vi) modulators (i.e., inhibitors, agonists and antagonists that alter the interaction between an over/underexpressed polypeptide and its binding partner. The dysfunctional antisense molecules are utilized to “knockout” endogenous function of a polypeptide by homologous recombination (see, e.g., Capecchi, Science 244: 1288-1292 1989). The term “siRNA” refers to a double stranded RNA molecule which prevents translation of a target mRNA. Standard techniques are used for introducing siRNA into cells, including those wherein DNA is used as the template to transcribe RNA. The siRNA comprises a sense nucleic acid sequence and an anti-sense nucleic acid sequence of the any one of the CML 1-190 gene. The siRNA is constructed such that a single transcript (double stranded RNA) has both the sense and complementary antisense sequences from the target gene, e.g., a hairpin.

Diseases and disorders that are characterized by decreased (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, a polypeptide (or analogs, derivatives, fragments or homologs thereof) or an agonist that increases bioavailability.

Increased or decreased levels can be readily detected by quantifying peptide and/or RNA, by obtaining a patient cell sample and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods includes contacting a cell with an agent that modulates one or more of the activities of the gene products of the differentially expressed genes. An agent that modulates protein activity includes a nucleic acid or a protein, a naturally-occurring cognate ligand of these proteins, a peptide, a peptidomimetic, or other small molecule. For example, the agent stimulates one or more protein activities of one or more of a differentially under-expressed gene.

The present invention also relates to a method of treating or preventing CML in a subject comprising administering to said subject a vaccine comprising a polypeptide encoded by a nucleic acid selected from the group consisting of CML 1-190 or an immunologically active fragment of said polypeptide, or a polynucleotide encoding the polypeptide or the fragment thereof. An administration of the polypeptide induce an anti-tumor immunity in a subject. To inducing anti-tumor immunity, a polypeptide encoded by a nucleic acid selected from the group consisting of CML 1-190 or an immunologically active fragment of said polypeptide, or a polynucleotide encoding the polypeptide is administered. The polypeptide or the immunologically active fragments thereof are useful as vaccines against CML. In some cases the proteins or fragments thereof may be administered in a form bound to the T cell receptor (TCR) or presented by an antigen presenting cell (APC), such as macrophage, dendritic cell (DC), or B-cells. Due to the strong antigen presenting ability of DC, the use of DC is most preferable among the APCs.

In the present invention, vaccine against CML refers to a substance that has the function to induce anti-tumor immunity upon inoculation into animals. According to the present invention, polypeptides encoded by CML 1-190 or fragments thereof were suggested to be HLA-A24 or HLA-A*0201 restricted epitopes peptides that may induce potent and specific immune response against CML cells expressing CML 1-190. Thus, the present invention also encompasses method of inducing anti-tumor immunity using the polypeptides. In general, anti-tumor immunity includes immune responses such as follows:
induction of cytotoxic lymphocytes against tumors,
induction of antibodies that recognize tumors, and
induction of anti-tumor cytokine production.

Therefore, when a certain protein induces any one of these immune responses upon inoculation into an animal, the protein is decided to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. A foreign substance that enters the living body is presented to T cells and B cells by the action of antigen presenting cells (APCs). T cells that respond to the antigen presented by APC in antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to T cell by APC, and detecting the induction of CTL. Furthermore, APC has the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTL using dendritic cells (DCs) as APC is well known in the art. DC is a representative APC having the strongest CTL inducing action among APCs. In this method, the test polypeptide is initially contacted with DC, and then this DC is contacted with T cells. Detection of T cells having cytotoxic effects against the cells of interest after the contact with DC shows that the test polypeptide has an activity of inducing the cytotoxic T cells. Activity of CTL against tumors can be detected, for example, using the lysis of 51Cr-labeled tumor cells as the indicator. Alternatively, the method of evaluating the degree of tumor cell damage using 3H-thymidine uptake activity or LDH (lactose dehydrogenase)-release as the indicator is also well known.

Apart from DC, peripheral blood mononuclear cells (PBMCs) may also be used as the APC. The induction of CTL is reported that the it can be enhanced by culturing PBMC in the presence of GM-CSF and IL-4. Similarly, CTL has been shown to be induced by culturing PBMC in the presence of keyhole limpet hemocyanin (KLH) and IL-7.

The test polypeptides confirmed to possess CTL inducing activity by these methods are polypeptides having DC activation effect and subsequent CTL inducing activity. Therefore, polypeptides that induce CTL against tumor cells are useful as vaccines against tumors. Furthermore, APC that acquired the ability to induce CTL against tumors by contacting with the polypeptides are useful as vaccines against tumors. Furthermore, CTL that acquired cytotoxicity due to presentation of the polypeptide antigens by APC can be also used as vaccines against tumors. Such therapeutic methods for tumors using anti-tumor immunity due to APC and CTL are referred to as cellular immunotherapy.

Generally, when using a polypeptide for cellular immunotherapy, efficiency of the CTL-induction is known to increase by combining a plurality of polypeptides having different structures and contacting them with DC. Therefore, when stimulating DC with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide can be determined to have an ability to induce anti-tumor immunity.

Anti-tumor immunity is induced by administering the vaccine of this invention, and the induction of anti-tumor immunity enables treatment and prevention of CNL. Therapy against cancer or prevention of the onset of cancer includes any of the steps, such as inhibition of the growth of cancerous cells, involution of cancer, and suppression of occurrence of cancer. Decrease in mortality of individuals having cancer, decrease of tumor markers in the blood, alleviation of detectable symptoms accompanying cancer, and such are also included in the therapy or prevention of cancer. Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA may be used for statistical analyses.

The above-mentioned protein having immunological activity or a vector encoding the protein may be combined with an adjuvant. An adjuvant refers to a compound that enhances the immune response against the protein when administered together (or successively) with the protein having immunological activity. Examples of adjuvants include cholera toxin, salmonella toxin, alum, and such, but are not limited thereto. Furthermore, the vaccine of this invention may be combined appropriately with a pharmaceutically acceptable carrier. Examples of such carriers are sterilized water, physiological saline, phosphate buffer, culture fluid, and such. Furthermore, the vaccine may contain as necessary, stabilizers, suspensions, preservatives, surfactants, and such. The vaccine is administered systemically or locally. Vaccine administration may be performed by single administration, or boosted by multiple administrations.

When using APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APC or CTL, the cells may be administered to the subject. APC can be also induced by introducing a vector encoding the polypeptide into PBMCs ex vivo. APC or CTL induced in vitro can be cloned prior to administration. By cloning and growing cells having high activity of damaging target cells, cellular immunotherapy can be performed more effectively. Furthermore, APC and CTL isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor immunity.

Pharmaceutical Compositions for Inhibiting CML

Pharmaceutical formulations include those suitable for oral, rectal, nasal, topical (including buccal and sub-lingual), vaginal or parenteral (including intramuscular, sub-cutaneous and intravenous) administration, or for administration by inhalation or insuflation. Preferably, administration is intravenous. The formulations are optionally packaged in discrete dosage units Pharmaceutical formulations suitable for oral administration include capsules, cachets or tablets, each containing a predetermined amount of the active ingredient. Formulations also include powders, granules or solutions, suspensions or emulsions. The active ingredient is optionally administered as a bolus electuary or paste. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein. A package of tablets may contain one tablet to be taken on each of the month. The formulation or dose of medicament varies with respect to the phase (chronic, accelerated, or blast crisis) of the CML.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration include suppositories with standard carriers such as cocoa butter or polyethylene glycol. Formulations for topical administration in the mouth, for example buccally or sublingually, include lozenges, which contain the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the compositions, e.g., polypeptides and organic compounds are administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity.

The invention will be further described in the following examples, which do not limit the scope of the invention described in the claims. The following examples illustrate the identification and characterization of genes differentially expressed in CML cells.

### EXAMPLE 1

### Preparation of Test Samples

Samples obtained from diseased cells and normal cells, e.g., mononuclear cells from peripheral blood, were evaluated to identify genes which are differently expressed in a disease state, e.g., CML. The assays were carried out as follows.

Patients and Samples

Peripheral blood samples were obtained from 27 CML patients prior to treatment with STI571. Each patient was then enrolled into a phase II study of STI571. To characterize CML cells, mRNA from 27 samples in which more than 65% of cells had been positive for the Ph chromosome prior to treatment by a FISH analysis detecting a bcr/abl fusion gene (13) were analyzed on a cDNA-microarray system. Of the 27, two cases were in accelerated phase and three cases were in blast crisis phase (Table 1). A mixture of mononuclear cells from peripheral blood from eleven healthy volunteers was used as a control.

TABLE 1
Clinicopathological features of patients examined

| Patient's | | | Ph (+) | |
|---|---|---|---|---|
| ID | Age (y) | Sex | (%) a | Phase |
| CML002 | 71 | F | 78 | Chronic |
| CML003 | 66 | M | 69 | Chronic |
| CML004 | 55 | F | 75.5 | Chronic |
| CML008 | 61 | F | 75 | Chronic |
| CML009 | 68 | M | 80.5 | Chronic |
| CML010 | 56 | M | 65.5 | Chronic |
| CML013 | 59 | F | 87 | Chronic |
| CML014 | 47 | M | 83.5 | Chronic |
| CML015 | 63 | F | 72 | Chronic |
| CML018 | 57 | M | 83.5 | Chronic |
| CML019 | 23 | M | 79 | Chronic |
| CML021 | 57 | M | 79.5 | Chronic |
| CML022 | 69 | M | 72.5 | Chronic |
| CML023 | 68 | F | 76 | Chronic |
| CML025 | 44 | M | 76 | Chronic |
| CML027 | 35 | M | 75.5 | Chronic |
| CML029 | 45 | F | 73 | Chronic |
| CML030 | 61 | M | 65.5 | Chronic |
| CML033 | 56 | M | 66 | Chronic |
| CML036 | 48 | M | 77 | Chronic |
| CML047 | 32 | F | 85.5 | Chronic |
| CML054 | 32 | M | 71 | Chronic |

RNA Preparation and T7-Based RNA Amplification

Mononuclear cells were prepared using Ficoll (Amersham Biosciences, Buckinghamshire, UK) and total RNA was extracted using TRIzol (Life Technologies, Inc., Grand Island, N.Y.) according to the manufacturer's instructions. After treatment with DNase I (Nippon Gene, Tokyo, Japan), T7-based RNA amplification was carried out (14). Two rounds of amplification using 2 μg of total RNA as starting material yielded 40-100 μg of amplified RNA (aRNA). For control samples from healthy volunteers, two rounds of T7-based RNA amplification was also performed to obtain a sufficient amount of aRNA. RNA amplified by this method accurately reflects the proportions in the original RNA source, as confirmed by semi-quantitative reverse transcription-polymerase chain reaction (RT-PCR) experiments, in which data from the microarrays were consistent with results from RT-PCR whether total RNA or amplified aRNA was used as the template (14).

Preparation of the cDNA Microarray and Hybridization

A genome-wide cDNA microarray was fabricated with 23,040 cDNAs selected from the UniGene database (build #131) of the National Center for Biotechnology Information (NCBI). To obtain cDNAs for spotting on the glass slides, RT-PCR was performed for each gene as described previously. (15) The PCR products were spotted on type 7 glass slides (Amersham Biosciences) by Microarray Spotter Generation III (Amersham Biosciences); 4,608 genes were spotted in duplicate on a single slide. Five different sets of slides (total 23,040 genes) were prepared, on each of which the same 52 housekeeping genes and two negative-control genes were spotted as well. Labeling, hybridization, washing, scanning, and quantification of signals were performed as described previously (14) except that all processes were carried out with an Automated Slide Processor (15).

Quantification of Signals and Data Analysis

The intensity of each hybridization signal was calculated photometrically using the ArrayVision computer program (Amersham Biosciences). Each slide contained 52 housekeeping genes, and the Cy5/Cy3 ratio for each gene's expression was adjusted so that the averaged Cy5/Cy3 ratio of the panel of housekeeping genes was 1.0. A cut-off value was assigned to each microarray slide, using variance analysis. If both Cy3 and Cy5 signal intensities were lower than the cut-off values, the expression level of the corresponding gene in that sample was assessed as absent. For other genes the Cy5/Cy3 ratio was calculated using raw data of each sample.

### EXAMPLE 2

### Identification of CML-Associated Genes

The relative expression ratio of each gene (Cy5/Cy3 intensity ratio) was classified into one of four categories: (1) highly up-regulated (expression ratio more than 5.0 in more than 50% of the informative cases); (2) highly down-regulated (expression ratio less than 0.2 in more than 50% of the informative cases); (3) low expression (expression ratio between 0.2 and 5.0 in more than 50% of the informative cases); and (4) not expressed (or slight expression but under the cut-off level for detection). These categories were used to detect a set of genes whose changes in expression ratios were common among samples as well as specific to a certain subgroup. To detect candidate genes that were commonly up- or down-regulated in CML cells, the overall expression patterns of 23,040 genes were screened to select genes with expression ratios of more than 5.0 or less than 0.2 that were present in more than 50% of chronic phase of the CML cases categorized as (1), (2), or (3).

Identification of Genes with Clinically Relevant Expression Patterns in CML Cells

The expression patterns of approximately 23,000 genes in CML cells were examined using cDNA microarray. Individual data was excluded when both Cy5 and Cy3 signals were under cut-off values. The computational analysis identified commonly highly up-regulated or down-regulated genes in CML cells; 190 genes revealed the expression ratio of>5.0 in more than 50% of informative cases and 106 genes showed the expression ratio of<0.2 in more than 50% of informative cases as down-regulated genes.

One hundred ninety genes were found to be highly up-regulated. The upregulated genes included genes encoding proteins involved in cell cycle regulation, growth promotion, and transcriptional activation and those having protein kinase activity. Many of them were shown to be over-expressed in other carcinomas. For example, MYB, a transcriptional activator that causes acute leukemia and transforms only hematopoietic cells (16), was highly expressed in over 90% of the chronic phase of CML cells. GATA-binding protein 2 (GATA2), also a transcriptional activator which regulates endotherin-1 gene expression in endothelial cells (17), was reported to be activated in 93% of acute myeloid leukemia (AML), 70% of acute lymphoblastic leukemia (ALL), and 83% of CML (18). In particular, 28 genes for example, ribonuclease RNase A family 3, (RNASE3), bactericidal/permeability-increasing protein (BP1), defensin alpha 1, myeloid-related sequence (DEFA1), aminolevulinate, delta-synthase 1 (ALAS1), elastase 2, neutrophil (ELA2), cathepsin G (CTSG), matrix metalloprotease 9 (MMP-9), haptoglobin-related protein (HPR), urokinase plasminogen activator, (UPLA), haptoglobin (HP), H3 histone family, member J (H3FJ), and hemoglobin, zeta (HBZ) were overexpressed in all of the informative samples in this study (see Table 3). MMP-9, an enzyme to degrade collagen type IV, is thought to be associated with the transmigration and degradation of the extracellular matrix structures of tissue and blood vessels. The expression of MMP-9 was enhanced in mononuclear cells of CML patients (19). Moreover, primary human Ph+ cells were reported to secrete various angiogenesis factors including MMP-9 (20). Thus, overexpression of MMP-9 might play an important role in the pathogenesis of CML. Furthermore, members in the hemoglobin family, for example, zeta (HBZ), beta (HBB), gamma G (HBG2), delta (HBD), and alpha 2 (HBA2) were overexpressed in more than 80% of the informative cases. In addition, haptoglobin (HP) and haptoglobin-related protein (HPR) also showed enhanced expression in all informative cases. Recent studies have shown that bcr/abl expression induced hemoglobin (Hb) production in HL-60/BCR-Abl cells or CML cells (21), (22). This suggests that the constitutively activated tyrosine kinase bcr-abl enhanced survival and expansion of hematopoietic progenitor cells.

One hundred six genes were found to be significantly down-regulated in the chronic phase of CML (see Table 4). The genes of known function included the SH3-domain GRB2-line 2 (SH3GL2), PCAF associated factor 65 beta (PAF65B), heparan sulfate 6-O-sulfotransferase (HS6ST), immunoglobulin heavy constant gamma 3 (IGHG3), heat shock 27 kD protein 2 (HSPB2), and prostaglandin D synthase gene (PTGDS) genes whose expression was suppressed in more than 90% of informative cases. A number of transcriptional negative regulators like DNA-dependent protein kinase catalytic subunit-interacting protein 2 (KIP2) were also included. KIP2 is a negative regulator of cell proliferation and arrests cells at the G1 phase. KIP2 was down-regulated in approximately 60% of the informative CML cases. Therefore, its down-regulation may confer continuous proliferative properties of leukemic cells.

Some of the significantly down-regulated genes reflect the difference in the cell types, the lymphocyte-specific genes such as genes encoding immune components, e.g., immunoglobulins, and complement component 2 (C2) as well as the markers for lymphocytes such as CD7, CD3E, CD79A, CD3Z, CD6, CD4, and CD79B antigens, interferon regulatory factor 4 (IRF4), and interleukin 7 receptor (IR7R) (see Table 4). The majority of the cells contained in the chronic phase of CML cells used in this study corresponded to blast cells of myeloid lineage. Although the peripheral white cell used as universal controls contained cells of both myeloid and lymphoid lineage, lymphocytes accounted for only a small population of the cells in the sample. This might represent that the population of lymphocyte in CML patients decreased compared with blood of healthy individuals.

TABLE 3
UP-REGULATED GENES

| | ratio(%) | informative | ratio > | Accession | | |
|---|---|---|---|---|---|---|
| CML Assignment | 1) | cases | 5 2) | No. | Symbol | Gene name |
| 1 | 100 | 22 | 22 | X16545 | RNASE3 | ribonuclease, RNase A family, |
| | | | | | | 3 (eosinophil cationic protein) |
| 2 | 100 | 22 | 22 | J04739 | BPI | bactericidal/permeability-increasing |
| | | | | | | protein |
| 3 | 100 | 22 | 22 | M21130 | DEFA1 | defensin, alpha 1, myeloid-related |
| | | | | | | sequence |
| 4 | 100 | 22 | 22 | X56351 | ALAS1 | aminolevulinate, delta-, synthase 1 |
| 5 | 100 | 21 | 21 | M16117 | CTSG | cathepsin G |
| 6 | 100 | 21 | 21 | M34379 | ELA2 | elastase 2, neutrophil |
| 7 | 100 | 20 | 20 | J05070 | MMP9 | matrix metalloproteinase 9 (gelatinase |
| | | | | | | B, 92 kD gelatinase, 92 kD |
| | | | | | | type IV collagenase) |
| 8 | 100 | 20 | 20 | K03431 | HPR | haptoglobin-related protein |
| 9 | 100 | 20 | 20 | F21002 | | ESTs |
| 10 | 100 | 19 | 19 | X17042 | PRG1 | proteoglycan 1, secretory granule |
| 11 | 100 | 19 | 19 | M14502 | ARG1 | arginase, liver |
| 12 | 100 | 18 | 18 | L06895 | MAD | MAX dimerization protein |
| 13 | 100 | 18 | 18 | M69199 | G0S2 | putative lymphocyte G0/G1 switch |
| | | | | | | gene |
| 14 | 100 | 17 | 17 | K01763 | HP | haptoglobin |
| 15 | 100 | 16 | 16 | H23213 | | ESTs |
| 16 | 100 | 16 | 16 | Z98744 | H3FJ | H3 histone family, member J |
| 17 | 100 | 15 | 15 | M24173 | HBZ | hemoglobin, zeta |
| 18 | 100 | 15 | 15 | X02419 | PLAU | plasminogen activator, urokinase |
| 19 | 100 | 15 | 15 | H48537 | | ESTs |
| 20 | 100 | 15 | 15 | AA191449 | KIAA1254 | KIAA1254 protein |
| 21 | 100 | 15 | 15 | AI022380 | | ESTs |
| 22 | 100 | 15 | 15 | T03595 | | Homo sapiens cDNA FLJ12688 |
| | | | | | | fis, clone NT2RM4002534 |
| 23 | 100 | 14 | 14 | X57129 | H1F2 | H1 histone family, member 2 |
| 24 | 100 | 14 | 14 | AA382504 | | ESTs |
| 25 | 100 | 13 | 13 | R26792 | GCL | grancalcin |
| 26 | 100 | 12 | 12 | AA815247 | | EST |
| 27 | 100 | 11 | 11 | M81637 | GCL | grancalcin |
| 28 | 100 | 11 | 11 | AA446449 | | EST |
| 29 | 95.45 | 22 | 21 | M83202 | LTF | lactotransferrin |
| 30 | 95.45 | 22 | 21 | V00497 | HBB | hemoglobin, beta |
| 31 | 95.45 | 22 | 21 | U01317 | HBD | hemoglobin, delta |
| 32 | 95.24 | 21 | 20 | AA489915 | HBG2 | hemoglobin, gamma G |
| 33 | 95 | 20 | 19 | AI023753 | | ESTs |
| 34 | 94.74 | 19 | 18 | X83006 | LCN2 | lipocalin 2 (oncogene 24p3) |
| 35 | 93.75 | 16 | 15 | AI040591 | | ESTs |
| 36 | 93.33 | 15 | 14 | AA855085 | NCOA4 | nuclear receptor coactivator 4 |
| 37 | 91.67 | 12 | 11 | M65085 | FSHR | follicle stimulating hormone receptor |
| 38 | 91.67 | 12 | 11 | AA398536 | | ESTs |
| 39 | 91.67 | 12 | 11 | AA843554 | | ESTs |
| 40 | 90.91 | 22 | 20 | S81914 | IER3 | immediate early response 3 |
| 41 | 90.91 | 22 | 20 | M27717 | CPA3 | carboxypeptidase A3 (mast cell) |
| 42 | 90.91 | 22 | 20 | U22376 | MYB | v-myb avian myeloblastosis viral |
| | | | | | | oncogene homolog |
| 43 | 90.91 | 11 | 10 | U32315 | STX3A | syntaxin 3A |
| 44 | 90.91 | 11 | 10 | AA774546 | NXF3 | nuclear RNA export factor 3 |
| 45 | 90.48 | 21 | 19 | AI015633 | | Solute carrier family 26, member 8 |
| 46 | 89.47 | 19 | 17 | M33987 | CA1 | carbonic anhydrase I |
| 47 | 88.89 | 18 | 16 | AA004412 | | ESTs |
| 48 | 88.89 | 18 | 16 | AI056326 | | ESTs |
| 49 | 88.24 | 17 | 15 | AA825819 | LOC55871 | COBW-like protein |
| 50 | 86.67 | 15 | 13 | X65614 | S100P | S100 calcium-binding protein P |
| 51 | 86.67 | 15 | 13 | L27711 | CDKN3 | cyclin-dependent kinase inhibitor |
| | | | | | | 3 (CDK2-associated dual specificity |
| | | | | | | phosphatase) |
| 52 | 86.67 | 15 | 13 | AA418448 | | ESTs |
| 53 | 86.67 | 15 | 13 | AA235222 | LOC51053 | geminin |
| 54 | 86.36 | 22 | 19 | M62831 | ETR101 | immediate early protein |
| 55 | 86.36 | 22 | 19 | L01664 | CLC | Charot-Leyden crystal protein |
| 56 | 86.36 | 22 | 19 | V00493 | HBA2 | hemoglobin, alpha 2 |
| 57 | 86.36 | 22 | 19 | X00637 | HP | haptoglobin |
| 58 | 86.36 | 22 | 19 | W63676 | | ESTs |
| 59 | 85.71 | 21 | 18 | U01317 | HBD | hemoglobin, delta |
| 60 | 85.71 | 14 | 12 | N52485 | DKFZP434O125 | DKFZP434O125 protein |
| 61 | 85.71 | 14 | 12 | AI335266 | FER1L3 | fer (C. elegans)-like 3 (myoferlin) |
| 62 | 85 | 20 | 17 | Z83821 | ALAS2 | aminolevulinate, delta-, synthase |
| | | | | | | 2 (sideroblastic/hypochromic |
| | | | | | | anemia) |
| 63 | 85 | 20 | 17 | AA318275 | FTH1 | Ferritin, heavy polypeptide 1 |
| 64 | 84.62 | 13 | 11 | M16827 | ACADM | acyl-Coenzyme A dehydrogenase, |
| | | | | | | C-4 to C2 straight chain |
| 65 | 83.33 | 12 | 10 | AA355657 | CTSG | cathepsin G |
| 66 | 81.82 | 22 | 18 | D14874 | ADM | adrenomedullin |
| 67 | 81.82 | 22 | 18 | M69043 | NFKBIA | nuclear factor of kappa light polypeptide |
| | | | | | | gene enhancer in |
| | | | | | | B-cells inhibitor, alpha |
| 68 | 81.82 | 22 | 18 | X55656 | HBG2 | hemoglobin, gamma G |
| 69 | 81.82 | 22 | 18 | X68277 | DUSP1 | dual specificity phosphatase 1 |
| 70 | 81.82 | 22 | 18 | W76477 | JUN | v-jun avian sarcoma virus 17 oncogene |
| | | | | | | homolog |
| 71 | 81.82 | 22 | 18 | X56351 | ALAS1 | aminolevulinate, delta-, synthase 1 |
| 72 | 81.82 | 11 | 9 | AI168658 | FECH | ferrochelatase (protoporphyria) |
| 73 | 81.82 | 11 | 9 | AA256671 | FLJ21939 | hypothetical protein FLJ21939 |
| | | | | | | similar to 5-azacytidine induced |
| | | | | | | gene 2 |
| 74 | 80 | 15 | 12 | AI819724 | COL1A1 | collagen, type I, alpha 1 |
| 75 | 78.95 | 19 | 15 | M96839 | PRTN3 | proteinase 3 (serine proteinase, |
| | | | | | | neutrophil, Wegener granulomatosis |
| | | | | | | autoantigen) |
| 76 | 78.95 | 19 | 15 | R78436 | GATA2 | GATA-binding protein 2 |
| 77 | 78.57 | 14 | 11 | AA449950 | KIAA1016 | KIAA1016 protein |
| 78 | 77.78 | 18 | 14 | AA456242 | FSP-2 | fibrousheathin II |
| 79 | 77.78 | 18 | 14 | AI357641 | CDKN2C | cyclin-dependent kinase inhibitor |
| | | | | | | 2C (p18, inhibits CDK4) |
| 80 | 77.78 | 18 | 14 | M13692 | ORM1 | orosomucoid 1 |
| 81 | 77.78 | 18 | 14 | D90145 | SCYA3L1 | small inducible cytokine A3-like 1 |
| 82 | 77.78 | 18 | 14 | AA903016 | HM74 | putative chemokine receptor; |
| | | | | | | GTP-binding protein |
| 83 | 77.27 | 22 | 17 | AI128538 | LOC51312 | mitochondrial solute carrier |
| 84 | 76.92 | 13 | 10 | AF041245 | HCRTR2 | hypocretin (orexin) receptor 2 |
| 85 | 76.47 | 17 | 13 | D13752 | CYP11B2 | cytochrome P450, subfamily |
| | | | | | | XIB (steroid 11-beta-hydroxylase), |
| | | | | | | polypeptide 2 |
| 86 | 76.47 | 17 | 13 | AI360412 | ALS2CR2 | Amyotrophic lateral sclerosis |
| | | | | | | 2 (juvenile) chromosome region, |
| | | | | | | candidate 2 |
| 87 | 76.47 | 17 | 13 | N30414 | | ESTs |
| 88 | 76.19 | 21 | 16 | AA126620 | C8FW | Phosphoprotein regulated by |
| | | | | | | mitogenic pathways |
| 89 | 75 | 20 | 15 | M33492 | TPSB1 | tryptase beta 1 |
| 90 | 75 | 20 | 15 | AF043584 | IGL | immunoglobulin lambda chain |
| 91 | 75 | 16 | 12 | AI000650 | | ESTs |
| 92 | 75 | 16 | 12 | AW337343 | PTP4A1 | protein tyrosine phosphatase type |
| | | | | | | IVA, member 1 |
| 93 | 73.68 | 19 | 14 | AA043835 | DAPP1 | dual adaptor of phosphotyrosine |
| | | | | | | and 3-phosphoinositides |
| 94 | 73.33 | 15 | 11 | AA449227 | | EST |
| 95 | 72.73 | 22 | 16 | U21847 | TIEG | TGFB inducible early growth response |
| 96 | 72.73 | 11 | 8 | U77942 | STX7 | syntaxin 7 |
| 97 | 72.73 | 11 | 8 | AA345854 | ITGA3 | integrin, alpha 3 (antigen CD4 |
| | | | | | | 9C, alpha 3 subunit of VLA-3 |
| | | | | | | receptor) |
| 98 | 72.73 | 11 | 8 | AA314457 | LOC56994 | cholinephosphotransferase 1 |
| 99 | 72.73 | 11 | 8 | AI718618 | BIRC2 | baculoviral IAP repeat-containing 2 |
| 100 | 72.73 | 11 | 8 | AI276054 | FRAT2 | Frequently rearranged in advanced |
| | | | | | | T-cell lymphomas 2 |
| 101 | 72.22 | 18 | 13 | D86724 | ARG2 | arginase, type II |
| 102 | 72.22 | 18 | 13 | X06233 | S100A9 | S100 calcium-binding protein |
| | | | | | | A9 (calgranulin B) |
| 103 | 72.22 | 18 | 13 | AA973757 | STX3A | syntaxin 3A |
| 104 | 72.22 | 18 | 13 | AF068754 | HSBP1 | heat shock factor binding protein 1 |
| 105 | 71.43 | 14 | 10 | H97976 | | ESTs |
| 106 | 70.59 | 17 | 12 | D14657 | KIAA0101 | KIAA0101 gene product |
| 107 | 70 | 20 | 14 | AI056641 | FLJ22833 | hypothetical protein FLJ22833 |
| 108 | 70 | 20 | 14 | AA677931 | | ESTs |
| 109 | 69.23 | 13 | 9 | AB003476 | AKAP12 | A kinase (PRKA) anchor protein |
| | | | | | | (gravin) 12 |
| 110 | 68.75 | 16 | 11 | AA854469 | RNF6 | ring finger protein (C3H2C3 type) 6 |
| 111 | 68.18 | 22 | 15 | AA327207 | | ESTs |
| 112 | 66.67 | 21 | 14 | AA772709 | | Homo sapiens cDNA FLJ13522 |
| | | | | | | fis, clone PLACE1005884 |
| 113 | 66.67 | 21 | 14 | U46254 | | ESTs |
| 114 | 66.67 | 21 | 14 | AI087002 | | ESTs |
| 115 | 66.67 | 18 | 12 | AW237266 | ASAH | N-acylsphingosine amidohydrolase |
| | | | | | | (acid ceramidase) |
| 116 | 66.67 | 12 | 8 | H53099 | NDUFA10 | NADH dehydrogenase (ubiquinone) |
| | | | | | | 1 alpha subcomplex, 10 |
| | | | | | | (42 kD) |
| 117 | 66.67 | 12 | 8 | T03044 | | EST |
| 118 | 65 | 20 | 13 | W96110 | ZNF281 | zinc finger protein 281 |
| 119 | 64.71 | 17 | 11 | AA629596 | DKFZP564D177 | DKFZP564D177 protein |
| 120 | 64.29 | 14 | 9 | M23161 | THE1 | Human transposon-like element |
| | | | | | | mRNA |
| 121 | 64.29 | 14 | 9 | D20186 | DKFZp762O076 | hypothetical protein DKFZp762O076 |
| 122 | 64.29 | 14 | 9 | H80325 | BAZ1A | bromodomain adjacent to zinc |
| | | | | | | finger domain, 1A |
| 123 | 63.64 | 22 | 14 | U21847 | TIEG | TGFB inducible early growth response |
| 124 | 63.64 | 11 | 7 | M31452 | C4BPA | complement component 4-binding |
| | | | | | | protein, alpha |
| 125 | 63.64 | 11 | 7 | U39231 | GIPR | gastric inhibitory polypeptide receptor |
| 126 | 63.64 | 11 | 7 | AI262031 | ATP10D | ATPase, Class V, type 10D |
| 127 | 63.64 | 11 | 7 | AF022385 | PDCD10 | programmed cell death 10 |
| 128 | 63.64 | 11 | 7 | N58488 | | EST |
| 129 | 63.64 | 11 | 7 | AI016419 | | ESTs |
| 130 | 62.5 | 16 | 10 | H71303 | KIAA0481 | KIAA0481 gene product |
| 131 | 62.5 | 16 | 10 | H06819 | FLJ10846 | hypothetical protein FLJ10846 |
| 132 | 62.5 | 16 | 10 | W93000 | | ESTs |
| 133 | 62.5 | 16 | 10 | W37916 | HCF-2 | host cell factor 2 |
| 134 | 61.9 | 21 | 13 | AF016833 | MGAM | maltase-glucoamylase (alpha- |
| | | | | | | glucosidase) |
| 135 | 61.54 | 13 | 8 | X00948 | RLN2 | relaxin 2 (H2) |
| 136 | 61.54 | 13 | 8 | U55206 | GGH | gamma-glutamyl hydrolase (conjugase, |
| | | | | | | folylpolygammaglutamyl |
| | | | | | | hydrolase) |
| 137 | 61.54 | 13 | 8 | AA308062 | S100P | S100 calcium-binding protein P |
| 138 | 61.54 | 13 | 8 | AA731746 | | ESTs |
| 139 | 60 | 20 | 12 | AI078178 | | ESTs |
| 140 | 60 | 15 | 9 | M95809 | GTF2H1 | general transcription factor II |
| | | | | | | H, polypeptide 1 (62 kD subunit) |
| 141 | 60 | 15 | 9 | AA401589 | | ESTs |
| 142 | 59.09 | 22 | 13 | M93056 | SERPINB1 | serine (or cysteine) proteinase |
| | | | | | | inhibitor, clade B (ovalbumin), |
| | | | | | | member 1 |
| 143 | 59.09 | 22 | 13 | AA436509 | IER5 | Immediate early response 5 |
| 144 | 58.82 | 17 | 10 | L10101 | SRY | sex determining region Y |
| 145 | 58.33 | 12 | 7 | W87690 | | Homo sapiens cDNA: FLJ23173 |
| | | | | | | fis, clone LNG10019 |
| 146 | 57.89 | 19 | 11 | AK025906 | | Homo sapiens cDNA: FLJ22253 |
| | | | | | | fis, clone HRC02763 |
| 147 | 57.89 | 19 | 11 | AA548765 | SMARCB1 | SWI/SNF related, matrix associated, |
| | | | | | | actin dependent regulator |
| | | | | | | of chromatin, subfamily b, member 1 |
| 148 | 57.14 | 21 | 12 | AI014551 | | ESTs |
| 149 | 57.14 | 21 | 12 | AA313441 | | Homo sapiens cDNA FLJ11838 |
| | | | | | | fis, clone HEMBA1006624, |
| | | | | | | weakly similar to DNA/PANTOTHENATE |
| | | | | | | METABOLISM |
| | | | | | | FLAVOPROTEIN HOMOLOG |
| 150 | 57.14 | 14 | 8 | D49958 | GPM6A | glycoprotein M6A |
| 151 | 57.14 | 14 | 8 | AA778025 | | ESTs |
| 152 | 57.14 | 14 | 8 | AA047169 | | Homo sapiens cDNA: FLJ22756 |
| | | | | | | fis, clone KAIA0791 |
| 153 | 56.25 | 16 | 9 | L16464 | ETV3 | ets variant gene 3 |
| 154 | 56.25 | 16 | 9 | AF011468 | STK15 | serine/threonine kinase 15 |
| 155 | 56.25 | 16 | 9 | AI188389 | C11ORF15 | chromosome 11 open reading frame |
| | | | | | | 15 |
| 156 | 55 | 20 | 11 | AA847136 | CSF2RB | Colony stimulating factor 2 receptor, |
| | | | | | | beta, low-affinity (granulocyte- |
| | | | | | | macrophage) |
| 157 | 55 | 20 | 11 | AI025297 | KLF7 | Kruppel-like factor 7 (ubiquitous) |
| 158 | 54.55 | 22 | 12 | M60974 | GADD45A | growth arrest and DNA-damage- |
| | | | | | | inducible, alpha |
| 159 | 54.55 | 22 | 12 | X56351 | ALAS1 | aminolevulinate, delta-, synthase 1 |
| 160 | 54.55 | 11 | 6 | U93869 | RPC39 | polymerase (RNA) III (DNA directed) |
| | | | | | | (39 kD) |
| 161 | 54.55 | 11 | 6 | AA551628 | FLJ10260 | hypothetical protein FLJ10260 |
| 162 | 54.55 | 11 | 6 | AI268502 | | ESTs |
| 163 | 54.55 | 11 | 6 | AI768505 | KIAA0707 | KIAA0707 protein |
| 164 | 54.55 | 11 | 6 | W70293 | | ESTs |
| 165 | 53.85 | 13 | 7 | M28443 | AMY2A | amylase, alpha 2A; pancreatic |
| 166 | 53.85 | 13 | 7 | L77571 | DGS-A | DiGeorge syndrome gene A |
| 167 | 53.85 | 13 | 7 | AA101229 | | ESTs |
| 168 | 53.33 | 15 | 8 | X51699 | BGLAP | bone gamma-carboxyglutamate |
| | | | | | | (gla) protein (osteocalcin) |
| 169 | 53.33 | 15 | 8 | R86067 | | ESTs, Weakly similar to KIAA1353 |
| | | | | | | protein [H. sapiens] |
| 170 | 53.33 | 15 | 8 | U57961 | 13CDNA73 | putative gene product |
| 171 | 53.33 | 15 | 8 | AI025822 | | EST |
| 172 | 53.33 | 15 | 8 | AI150469 | | ESTs |
| 173 | 52.94 | 17 | 9 | M23204 | OAT | ornithine aminotransferase (gyrate |
| | | | | | | atrophy) |
| 174 | 52.63 | 19 | 10 | L04270 | LTBR | lymphotoxin beta receptor (TNFR |
| | | | | | | superfamily, member 3 |
| 175 | 52.63 | 19 | 10 | AW511361 | SLC29A1 | solute carrier family 29 (nucleoside |
| | | | | | | transporters), member 1 |
| 176 | 52.63 | 19 | 10 | AA027229 | | ESTs, Weakly similar to F45 |
| | | | | | | E12.5 [C. elegans] |
| 177 | 52.63 | 19 | 10 | AI201982 | | Homo sapiens cDNA FLJ1151 |
| | | | | | | 6 fis, clone HEMBA1002328 |
| 178 | 52.38 | 21 | 11 | AA706319 | | ESTs |
| 179 | 52.38 | 21 | 11 | W05688 | | ESTs |
| 180 | 50 | 20 | 10 | X56741 | MEL | mel transforming oncogene (derived |
| | | | | | | from cell line NK14)-R |
| | | | | | | AB8 homolog |
| 181 | 50 | 18 | 9 | AI032402 | | ESTs |
| 182 | 50 | 16 | 8 | F10728 | | ESTs |
| 183 | 50 | 16 | 8 | AA576399 | | ESTs |
| 184 | 50 | 16 | 8 | T36260 | SEC23B | Sec23 (S. cerevisiae) homolog B |
| 185 | 50 | 16 | 8 | AJ250075 | CH1 | membrane protein CH1 |
| 186 | 50 | 12 | 6 | AI187066 | | ESTs |
| 187 | 50 | 12 | 6 | AA586974 | PI3 | protease inhibitor 3, skin-derived |
| | | | | | | (SKALP) |
| 188 | 50 | 14 | 7 | AA639599 | SLC12A2 | solute carrier family 12 (sodium/ |
| | | | | | | potassium/chloride transporters), |
| | | | | | | member 2 |
| 189 | 50 | 14 | 7 | AA807551 | | ESTs |
| 190 | 50 | 14 | 7 | AA503224 | | ESTs |

Accession numbers and gene symbols were retrieved from the Unigene Databases (build#131).

TABLE 4
DOWN-REGULATED GENES

| CML | ratio(%) | informative | ratio < | Accession | | |
|---|---|---|---|---|---|---|
| Assignment | 1) | cases | 5 2) | No. | Symbol | Gene name |
| 191 | 100 | 16 | 16 | AF036268 | SH3GL2 | SH3-domain GRB2-like 2 |
| 192 | 94.74 | 19 | 18 | AF069736 | PAF65B | PCAF associated factor 65 beta |
| 193 | 93.75 | 16 | 15 | AB006179 | HS6ST | heparan sulfate 6-O-sulfotransferase |
| 194 | 93.33 | 15 | 14 | AA806043 | IGHG3 | immunoglobulin heavy constant gamma |
| | | | | | | 3 (G3m marker) |
| 195 | 90.91 | 11 | 10 | D89617 | HSPB2 | heat shock 27 kD protein 2 |
| 196 | 90.48 | 21 | 19 | M61900 | PTGDS | prostaglandin D synthase gene |
| 197 | 88.24 | 17 | 15 | M73780 | ITGB8 | integrin, beta 8 |
| 198 | 88.24 | 17 | 15 | M60450 | KCNA4 | potassium voltage-gated channel, shaker- |
| | | | | | | related subfamily, member 4 |
| 199 | 87.5 | 16 | 14 | U28369 | SEMA3B | sema domain, immunoglobulin domain |
| | | | | | | (Ig), short basic domain, secreted, |
| | | | | | | (semaphorin) 3B |
| 200 | 86.67 | 15 | 13 | M13149 | HRG | histidine-rich glycoprotein |
| 201 | 86.67 | 15 | 13 | AI089023 | FXYD7 | FXYD domain-containing ion transport |
| | | | | | | regulator 7 |
| 202 | 86.36 | 22 | 19 | D00749 | CD7 | CD7 antigen |
| 203 | 85 | 20 | 17 | M88468 | MVK | mevalonate kinase (mevalonic aciduria) |
| 204 | 84.62 | 13 | 11 | AF040723 | HAP1 | huntingtin-associated protein 1 (neuroan |
| | | | | | | 1) |
| 205 | 84.21 | 19 | 16 | M24405 | TCF3 | transcription factor 3 (E2A immunoglobulin |
| | | | | | | enhancer binding factors |
| | | | | | | E12/E47) |
| 206 | 82.35 | 17 | 14 | M81886 | GRIA1 | glutamate receptor, ionotropic, AMPA 1 |
| 207 | 82.35 | 17 | 14 | M25809 | ATP6B1 | ATPase, H+ transporting, lysosomal |
| | | | | | | (vacuolar proton pump), beta polypeptide, |
| | | | | | | 56/58 kD, isoform 1 |
| 208 | 81.82 | 22 | 18 | R72651 | | ESTs, Weakly similar to PLK_HUMAN |
| | | | | | | PROTEOGLYCAN LINK |
| | | | | | | PROTEIN PRECURSOR [H. sapiens] |
| 209 | 81.82 | 22 | 18 | X97229 | KIR2DL4 | killer cell immunoglobulin-like receptor, |
| | | | | | | two domains, long cytoplasmic |
| | | | | | | tail, 4 |
| 210 | 81.82 | 22 | 18 | D87465 | KIAA0275 | KIAA0275 gene product |
| 211 | 81.82 | 11 | 9 | S77410 | AGTR1 | angiotensin receptor 1 |
| 212 | 81.82 | 11 | 9 | U52112 | ARD1 | N-acetyltransferase, homolog of S. cerevisiae |
| | | | | | | ARD1 |
| 213 | 81.25 | 16 | 13 | U18468 | PSG4 | pregnancy specific beta-glycoprotein 4 |
| 214 | 80 | 20 | 16 | D29990 | SLC7A2 | solute carrier family 7 (cationic amino |
| | | | | | | acid transporter, y+ system), |
| | | | | | | member 2 |
| 215 | 80 | 15 | 12 | M23323 | CD3E | CD3E antigen, epsilon polypeptide |
| 216 | 78.95 | 19 | 15 | M30607 | ZFY | zinc finger protein, Y-linked |
| 217 | 78.95 | 19 | 15 | M98833 | FLI1 | Friend leukemia virus integration 1 |
| 218 | 77.27 | 22 | 17 | X67292 | IGHM | immunoglobulin heavy constant mu |
| 219 | 76.92 | 13 | 10 | AA543086 | | Homo sapiens cDNA: FLJ23270 fis, |
| | | | | | | clone COL10309, highly similar |
| | | | | | | to HSU33271 Human normal keratinocyte |
| | | | | | | mRNA |
| 220 | 75 | 20 | 15 | L13258 | SLC34A1 | solute carrier family 34 (sodium phosphate), |
| | | | | | | member 1 |
| 221 | 75 | 12 | 9 | D00174 | SERPINF2 | serine (or cysteine) proteinase inhibitor, |
| | | | | | | clade F (alpha-2 antiplasmin, |
| | | | | | | pigment epithelium derived factor), |
| | | | | | | member 2 |
| 222 | 73.68 | 19 | 14 | U05227 | SEC4L | GTP-binding protein homologous |
| | | | | | | to Saccharomyces cerevisiae SEC4 |
| 223 | 73.33 | 15 | 11 | U79240 | | Human PAS-serine/threonine kinase |
| | | | | | | mRNA, partial cds |
| 224 | 73.33 | 15 | 11 | X07994 | LCT | lactase |
| 225 | 72.73 | 22 | 16 | M80462 | CD79A | CD79A antigen (immunoglobulin- |
| | | | | | | associated alpha) |
| 226 | 71.43 | 21 | 15 | J04132 | CD3Z | CD3Z antigen, zeta polypeptide (T |
| | | | | | | iT3 complex) |
| 227 | 70.59 | 17 | 12 | U97145 | GFRA2 | GDNF family receptor alpha 2 |
| 228 | 69.23 | 13 | 9 | U05321 | SLC16A2 | solute carrier family 16 (monocarboxylic |
| | | | | | | acid transporters), member |
| | | | | | | 2 (putative transporter) |
| 229 | 69.23 | 13 | 9 | M24405 | TCF3 | transcription factor 3 (E2A immunoglobulin |
| | | | | | | enhancer binding factors |
| | | | | | | E12/E47) |
| 230 | 69.23 | 13 | 9 | U02619 | GTF3C1 | general transcription factor IIIC, polypeptide |
| | | | | | | 1 (alpha subunit, 220 kD) |
| 231 | 68.75 | 16 | 11 | J04599 | BGN | biglycan |
| 232 | 68.42 | 19 | 13 | X80818 | GRM4 | glutamate receptor, metabotropic 4 |
| 233 | 68.42 | 19 | 13 | X70991 | NAB2 | NGFI-A binding protein 2 (ERG1 |
| | | | | | | binding protein 2) |
| 234 | 68.42 | 19 | 13 | J00269 | KRT6A | keratin 6A |
| 235 | 68.18 | 22 | 15 | AA778161 | RPL26 | ribosomal protein L26 |
| 236 | 66.67 | 18 | 12 | AI123516 | | ESTs |
| 237 | 66.67 | 15 | 10 | U34623 | CD6 | CD6 antigen |
| 238 | 66.67 | 12 | 8 | AA421322 | IGL@ | immunoglobulin lambda locus |
| 239 | 66.67 | 12 | 8 | M91196 | ICSBP1 | interferon consensus sequence binding |
| | | | | | | protein 1 |
| 240 | 66.67 | 12 | 8 | M12807 | CD4 | CD4 antigen (p55) |
| 241 | 66.67 | 12 | 8 | D52745 | KIAA0821 | lectomedin-2 |
| 242 | 64.29 | 14 | 9 | S82807 | TSHR | thyroid stimulating hormone receptor |
| 243 | 64.29 | 14 | 9 | AI027554 | DKFZP586J1624 | DKFZP586J1624 protein |
| 244 | 63.64 | 22 | 14 | U52682 | IRF4 | interferon regulatory factor 4 |
| 245 | 63.64 | 22 | 14 | X03066 | HLA-DOB | major histocompatibility complex, |
| | | | | | | class II, DO beta |
| 246 | 63.64 | 22 | 14 | M15800 | MAL | mal, T-cell differentiation protein |
| 247 | 63.64 | 22 | 14 | AI366242 | | ESTs |
| 248 | 63.64 | 22 | 14 | X54101 | GNLY | granulysin |
| 249 | 63.64 | 22 | 14 | T04932 | | Homo sapiens cDNA: FLJ21545 fis, |
| | | | | | | clone COL06195 |
| 250 | 63.64 | 22 | 14 | AI248183 | PAX5 | Paired box gene 5 (B-cell lineage specific |
| | | | | | | activator protein) |
| 251 | 63.64 | 11 | 7 | AF064804 | SUPT3H | suppressor of Ty (S. cerevisiae) 3 homolog |
| 252 | 63.64 | 11 | 7 | D28769 | PBX2 | pre-B-cell leukemia transcription factor 2 |
| 253 | 63.64 | 11 | 7 | AA620287 | | ESTs |
| 254 | 61.9 | 21 | 13 | X62071 | CDK2 | cyclin-dependent kinase 2 |
| 255 | 61.9 | 21 | 13 | AI271678 | | ESTs |
| 256 | 61.9 | 21 | 13 | X82240 | TCL1A | T-cell leukemia/lymphoma 1A |
| 257 | 61.54 | 13 | 8 | X78677 | KHK | ketohexokinase (fructokinase) |
| 258 | 61.11 | 18 | 11 | M75106 | CPB2 | carboxypeptidase B2 (plasma) |
| 259 | 59.09 | 22 | 13 | M74161 | INPP5B | inositol polyphosphate-5-phosphatase, |
| | | | | | | 75 kD |
| 260 | 59.09 | 22 | 13 | AI214175 | KIAA0655 | huntingtin interacting protein-related |
| 261 | 58.82 | 17 | 10 | X15218 | SKI | v-ski avian sarcoma viral oncogene |
| | | | | | | homolog |
| 262 | 57.89 | 19 | 11 | AA252866 | KIP2 | DNA-dependent protein kinase catalytic |
| | | | | | | subunit-interacting protein 2 |
| 263 | 57.14 | 21 | 12 | L19711 | DAG1 | dystroglycan 1 (dystrophin-associated |
| | | | | | | glycoprotein 1) |
| 264 | 55.56 | 18 | 10 | AA648810 | VCP | Valosin-containing protein |
| 265 | 55 | 20 | 11 | L13203 | FOXI1 | forkhead box I1 |
| 266 | 55 | 20 | 11 | U14534 | NR1H2 | nuclear receptor subfamily 1, group |
| | | | | | | H, member 2 |
| 267 | 54.55 | 22 | 12 | M29696 | IL7R | interleukin 7 receptor |
| 268 | 54.55 | 22 | 12 | M14745 | BCL2 | B-cell CLL/lymphoma 2 |
| 269 | 54.55 | 22 | 12 | AI341482 | RNB6 | RNB6 |
| 270 | 54.55 | 11 | 6 | U79255 | APBA2 | amyloid beta (A4) precursor protein- |
| | | | | | | binding, family A, member 2 (X11- |
| | | | | | | like) |
| 271 | 54.55 | 11 | 6 | U22526 | LSS | lanosterol synthase (2,3-oxidosqualene- |
| | | | | | | lanosterol cyclase) |
| 272 | 54.55 | 11 | 6 | S59049 | RGS1 | regulator of G-protein signalling 1 |
| 273 | 54.55 | 11 | 6 | M83651 | GALGT | UDP-N-acetyl-alpha-D-galactosamine: |
| | | | | | | (N-acetylneuraminyl)-galactosylglucosylceramide |
| | | | | | | N-acetylgalactosaminyltransferase |
| | | | | | | (GalNAc- |
| | | | | | | T) |
| 274 | 53.85 | 13 | 7 | L02867 | HUMPPA | paraneoplastic antigen |
| 275 | 53.85 | 13 | 7 | M35533 | LBP | lipopolysaccharide-binding protein |
| 276 | 53.85 | 13 | 7 | AI015930 | STMN3 | Stathmin-like 3 |
| 277 | 53.85 | 13 | 7 | U11276 | KLRB1 | killer cell lectin-like receptor subfamily |
| | | | | | | B, member 1 |
| 278 | 53.33 | 15 | 8 | M89957 | CD79B | CD79B antigen (immunoglobulin- |
| | | | | | | associated beta) |
| 279 | 52.63 | 19 | 10 | AL009179 | H3FK | H3 histone family, member K |
| 280 | 52.38 | 21 | 11 | D89618 | KPNA3 | karyopherin alpha 3 (importin alpha |
| | | | | | | 4) |
| 281 | 50 | 22 | 11 | M87790 | IGL λ | immunoglobulin lambda locus |
| 282 | 50 | 22 | 11 | X72475 | IGKC | immunoglobulin kappa constant |
| 283 | 50 | 22 | 11 | AF037261 | SCAM | vinexin beta (SH3-containing adaptor |
| | | | | | | molecule) |
| 284 | 50 | 22 | 11 | M17016 | GZMB | granzyme B (granzyme 2, cytotoxic |
| | | | | | | T-lymphocyte-associated serine |
| | | | | | | esterase 1) |
| 285 | 50 | 22 | 11 | AA813912 | KIAA0130 | KIAA0130 gene product |
| 286 | 50 | 22 | 11 | AI366182 | | ESTs |
| 287 | 50 | 20 | 10 | L31801 | SLC16A1 | solute carrier family 16 (monocarboxylic |
| | | | | | | acid transporters), member 1 |
| 288 | 50 | 20 | 10 | D26309 | LIMK1 | LIM domain kinase 1 |
| 289 | 50 | 18 | 9 | X68149 | BLR1 | Burkitt lymphoma receptor 1, GTP- |
| | | | | | | binding protein |
| 290 | 50 | 14 | 7 | D86479 | AEBP1 | AE-binding protein 1 |
| 291 | 50 | 14 | 7 | M88338 | MSE55 | serum constituent protein |
| 292 | 50 | 14 | 7 | U73036 | IRF7 | interferon regulatory factor 7 |
| 293 | 50 | 14 | 7 | AI336233 | | ESTs, Weakly similar to carnitine/ |
| | | | | | | acylcarnitine translocase [H. sapiens] |
| 294 | 50 | 12 | 6 | M19713 | TPM1 | tropomyosin 1 (alpha) |
| 295 | 50 | 12 | 6 | AJ002309 | SYNGR3 | synaptogyrin 3 |
| 296 | 50 | 12 | 6 | M73531 | RDS | retinal degeneration, slow (retinitis |
| | | | | | | pigmentosa 7) |

Accession numbers and gene symbols were retrieved from the Unigene Databases (build#131).

Confirmation by Semi-Quantitative RT-PCR

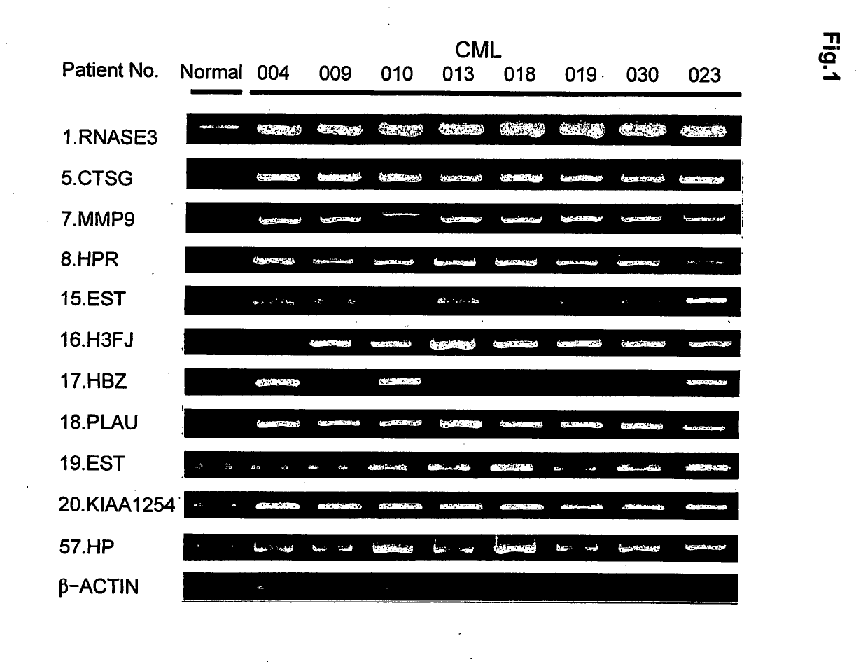

To confirm the reliability of the expression differences indicated by microarray analysis, semi-quantitative RT-PCR experiments were performed for the 11 highly up-regulated genes in all of the informative samples (RNASE3, CTSG, MMP9, HP, HPR, H3FJ, HBZ, PLAU, KIAA1254, and two ESTs (Accession No. H23213 and H48537). A 3-μg aliquot of aRNA from each sample was reverse-transcribed for single-stranded cDNAs using random primer (Roche) and Superscript II (Life Technologies, Inc.). Each cDNA mixture was diluted for subsequent PCR amplification with the same primer sets that were prepared for the target DNA- or β-actin-specific reactions. The primer sequences are listed in Table 2. Expression of β-actin served as an internal control. PCR reactions were optimized for the number of cycles to ensure product intensity within the linear phase of amplification. The RT-PCR results were highly concordant to those of the microarray analysis in the great majority of the tested cases (see FIG. 1). These data verified the reliability of our strategy to identify commonly up-regulated genes in CML cells.

TABLE 2

Primer sequences for semi-quantitative RT-PCR experiments

| | | |
|---|---|---|
| CML | SEQ. | SEQ. |

| | | | | | |
|---|---|---|---|---|---|
| Assign- | Accession | Forward | ID. | Reverse | ID. |

| | | | | | | |
|---|---|---|---|---|---|---|
| ment | No. | Symbol | primer | NO. | primer | NO. |

| | | | | | | |
|---|---|---|---|---|---|---|
| 1 | X16545 | RNASE3 | 5′-GTTCCAAAA | No. 1 | 5′-GGTATGGAGA | No. 2 |
| | | | CTGTTCACTTCC | | CTGATGAGGACA | |
| | | | C-3′ | | G-3′ | |
| 5 | M16117 | CTSG | 5′-CTTCTGCTGG | No. 3 | 5′-TGTGGACGTT | No. 4 |
| | | | CCTTTCTCCTA | | TATTAAGGCTCT | |
| | | | C-3′ | | G-3′ | |
| 7 | J05070 | MMP9 | 5′-GAACCAGCT | No. 5 | 5′-AAAACAAAGG | No. 6 |
| | | | GTATTTGTTCA | | TGAGAAGAGAG | |
| | | | AGG-3′ | | GG-3′ | |
| 8 | K03431 | HPR | 5′-TCCTGAATGT | No. 7 | 5′-AGCCTTGCAT | No. 8 |
| | | | GAAGCAGTATG | | TAGTTCTCAGCT | |
| | | | TG-3′ | | A-3′ | |
| 15 | H23213 | EST | 5′-GTCCCAAGA | No. 9 | 5′-CCGAGCCCAT | No. 10 |
| | | | TGCATATTTTCC | | TAATACTGATAG | |
| | | | T-3′ | | A-3′ | |
| 16 | Z98744 | H3FJ | 5′-ACTTTCTGAC | No. 11 | 5′-ACAGAGTGCT | No. 12 |
| | | | TTAGGCCACAG | | CAGTTCTTCCGT | |
| | | | GT-3′ | | A-3′ | |
| 17 | M24173 | HBZ | 5′-TCTCTGACCA | No. 13 | 5′-GAGGATACGA | No. 14 |
| | | | AGACTGAGAGG | | CCGATAGGAACT | |
| | | | AC-3′ | | T-3′ | |
| 18 | X02419 | PLAU | 5′-CAGTCACAC | No. 15 | 5′-CAGTGAGGAT | No. 16 |
| | | | CAAGGAAGAG | | TGGATGAACTAG | |
| | | | AATG-3′ | | G-3′ | |
| 19 | H48537 | EST | 5′-GTGTGATTAT | No. 17 | 5′-AATAGTGCCT | No. 18 |
| | | | CAAAAGGGAGT | | ATTTAAGGCC | |
| | | | GG-3′ | | G-3′ | |
| 20 | AA191449 | KIAA1254 | 5′-TCCTACTTTG | No. 19 | 5′-ACTAAGCTGG | No. 20 |
| | | | GCCAAGTTTGT | | TACATGGAATGG | |
| | | | T-3′ | | A-3′ | |
| 57 | K01763 | HP | 5′-AAGGAGATG | No. 21 | 5′-TGATTGACTC | No. 22 |
| | | | GAGTGTACACC | | AGCAATGCAG | |
| | | | TTAAA-3′ | | G-3′ | |
| | V00478 | ACTB | 5′-CATCCACGA | No. 23 | 5′-TCTCCTTAGA | No. 24 |
| | | | AACTACCTTCA | | GAGAAGTGGGG | |
| | | | ACT-3′ | | TG-3′ | |

### INDUSTRIAL APPLICABILITY

The gene-expression analysis of CML described herein, obtained through a combination of laser-capture dissection and genome-wide cDNA microarray, has identified specific genes as targets for cancer prevention and therapy. Based on the expression of a subset of these differentially expressed genes, the present invention provides a molecular diagnostic markers for identifying or detecting CML.

The methods described herein are also useful in the identification of additional molecular targets for prevention, diagnosis and treatment of CML. The data reported herein add to a comprehensive understanding of CML, facilitate development of novel diagnostic strategies, and provide clues for identification of molecular targets for therapeutic drugs and preventative agents. Such information contributes to a more profound understanding of tumorigenesis of CML, and provide indicators for developing novel strategies for diagnosis, treatment, and ultimately prevention of CML.

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

### REFERENCES

1. Faderl, S., Talpaz, M., Estrov, Z., O'Brien, S., Kurzrock, R., and Kantarjian, H. M. The biology of chronic myeloid leukemia. N Engl J Med, 341: 164-172., 1999.
2. Sawyers, C. L. Chronic myeloid leukemia N Engl J Med, 340: 1330-1340., 1999.
3. Silver, R. T., Woolf, S. H., Hehlmann, R., Appelbaum, F. R., Anderson, J., Bennett, C., Goldman, J. M., Guilhot, F., Kantarjian, H. M., Lichtin, A. E., Talpaz, M., and Tura, S. An evidence-based analysis of the effect of busulfan, hydroxyurea, interferon, and allogeneic bone marrow transplantation in treating the chronic phase of chronic myeloid leukemia: developed for the American Society of Hematology. Blood, 94: 1517-1536., 1999.
4. Buchdunger, E., Matter, A., and Druker, B. J. Bcr-Abl inhibition as a modality of CML therapeutics. Biochim Biophys Acta, 1551: M11-18., 2001.
5. Druker, B. J., Tamura, S., Buchdunger, E., Ohno, S., Segal, G. M., Fanning, S., Zimmermann, J., and Lydon, N. B. Effects of a selective inhibitor of the Abl tyrosine kinase on the growth of Bcr-Abl positive cells. Nat Med, 2: 561-566., 1996.
6. Druker, B. J., Sawyers, C. L., Kantarjian, H., Resta, D. J., Reese, S. F., Ford, J. M., Capdeville, R., and Talpaz, M. Activity of a specific inhibitor of the BCR-ABL tyrosine kinase in the blast crisis of chronic myeloid leukemia and acute lymphoblastic leukemia with the Philadelphia chromosome. N Engl J Med, 344: 1038-1042., 2001.
7. Sawyers, C. L., Hochhaus, A., Feldman, E., Goldman, J. M., Miller, C. B., Ottmann, O. G., Schiffer, C. A., Talpaz, M., Guilhot, F., Deininger, M. W., Fischer, T., O'Brien, S. G., Stone, R. M., Gambacorti-Passerini, C. B., Russell, N. H., Reiffers, J. J., Shea, T. C., Chapuis, B., Coutre, S., Tura, S., Morra, E., Larson, R. A., Saven, A., Peschel, C., Gratwohl, A., Mandelli, F., Ben-Am, M., Gathmann, I., Capdeville, R., Paquette, R. L., and Druker, B. J. Imatinib induces hematologic and cytogenetic responses in patients with chronic myelogenous leukemia in myeloid blast crisis: results of a phase II study.

Blood, 99: 3530-3539., 2002.
8. Gorre, M. E., Mohammed, M., Ellwood, K., Hsu, N., Paquette, R., Rao, P. N., and Sawyers, C. L. Clinical resistance to STI-571 cancer therapy caused by BCR-ABL gene mutation or amplification. Science, 293: 876-880., 2001.
9. von Bubnoff, N., Schneller, F., Peschel, C., and Duyster, J. BCR-ABL gene mutations in relation to clinical resistance of Philadelphia-chromosome-positive leukaemia to STI571: a prospective study. Lancet, 359: 487-491., 2002.
10. Armstrong, S. A., Staunton, J. E., Silverman, L. B., Pieters, R., den Boer, M. L., Minden, M. D., Sallan, S. E., Lander, E. S., Golub, T. R., and Korsmeyer, S. J. MLL translocations specify a distinct gene expression profile that distinguishes a unique leukemia Nat Genet, 30: 41-47., 2002.
11. Golub, T. R., Slonim, D. K, Tamayo, P., Huard, C., Gaasenbeek, M., Mesirov, J. P., Coller, H., Loh, M. L., Downing, J. R., Caligiuri, M. A., Bloomfield, C. D., and Lander, E. S. Molecular classification of cancer: class discovery and class prediction by gene expression monitoring. Science, 286: 531-537., 1999.
12. Hofmann, W. K., de Vos, S., Elashoff, D., Gschaidmeier, H., Hoelzer, D., Koeffler, H. P., and Ottmann, O. G. Relation between resistance of Philadelphia-chromosome-positive acute lymphoblastic leukaemia to the tyrosine kinase inhibitor STI571 and gene-expression profiles: a gene-expression study. Lancet, 359: 481-486., 2002.
13. Yanagi, M., Shinjo, K., Takeshita, A., Tobita, T., Yano, K., Kobayashi, M., Terasaki, H., Naoe, T., Ohnishi, K., and Ohno, R. Simple and reliably sensitive diagnosis and monitoring of Philadelphia chromosome-positive cells in chronic myeloid leukemia by interphase fluorescence in situ hybridization of peripheral blood cells. Leukemia, 13: 542-552., 1999.
14. Ono, K., Tanaka, T., Tsunoda, T., Kitahara, O., Kihara, C., Okamoto, A., Ochiai, K., Takagi, T., and Nakamura, Y. Identification by cDNA microarray of genes involved in ovarian carcinogenesis. Cancer Res, 60: 5007-5011., 2000.
15. Okabe, H., Satoh, S., Kato, T., Kitahara, O., Yanagawa, R., Yamaoka, Y., Tsunoda, T., Furukawa, Y., and Nakamura, Y. Genome-wide analysis of gene expression in human hepatocellular carcinomas using cDNA microarray: identification of genes involved in viral carcinogenesis and tumor progression. Cancer Res, 61: 2129-2137., 2001.
16. Lipsick, J. S. and Wang, D. M. Transformation by v-Myb. Oncogene, 18: 3047-3055., 1999.
17. Nagai, T., Harigae, H., Ishihara, H., Motohashi, H., Minegishi, N., Tsuchiya, S., Hayashi, N., Gu, L., Andres, B., Engel, J. D., and et al. Transcription factor GATA-2 is expressed in erythroid, early myeloid, and CD34+ human leukemia-derived cell lines. Blood, 84: 1074-1084., 1994.
18. Wang, L., Dong, L., Liu, G., and et al. [GATA-2 gene expression in leukemia patients and its significance]. Zhonghua Xue Ye Xue Za Zhi, 22: 27-29., 2001.
19. Ries, C., Loher, F., Zang, C., Ismair, M. G., and Petrides, P. E. Matrix metalloproteinase production by bone marrow mononuclear cells from normal individuals and patients with acute and chronic myeloid leukemia or myelodysplastic syndromes. Clin Cancer Res, 5: 1115-1124., 1999.
20. Janowska-Wieczorek, A., Majka, M., Marquez-Curtis, L., Wertheim, J. A., Turner, A. R., and Ratajczak, M. Z. Bcr-abl-positive cells secrete angiogenic factors including matrix metalloproteinases and stimulate angiogenesis in vivo in Matrigel implants. Leukemia, 16: 1160-1166., 2002.
21. Fang, G., Kim, C. N., Perkins, C. L., Ramadevi, N., Winton, E., Wittmann, S., and Bhalla, K N. CGP57148B (STI-571) induces differentiation and apoptosis and sensitizes Bcr-Abl-positive human leukemia cells to apoptosis due to antileukemic drugs. Blood, 96: 2246-2253., 2000.
22. Ghaffari, S., Kitidis, C., Fleming, M. D., Neubauer, H., Pfeffer, K., and Lodish, H. F. Erythropoiesis in the absence of janus-kinase 2: BCR-ABL induces red cell formation in JAK2(−/−) hematopoietic progenitors. Blood, 98: 2948-2957., 2001.
23. Druker, B. J., Talpaz, M., Resta, D. J., Peng, B., Buchdunger, E., Ford, J. M., Lydon, N. B., Kantarjian, H., Capdeville, R., Ohno-Jones, S., and Sawyers, C. L. Efficacy and safety of a specific inhibitor of the BCR-ABL tyrosine kinase in chronic myeloid leukemia N Engl J Med, 344: 1031-1037., 2001.
24. Kantarjian, H., Sawyers, C., Hochhaus, A., Guilhot, F., Schiffer, C., Gambacorti-Passerini, C., Niederwieser, D., Resta, D., Capdeville, R., Zoellner, U., Talpaz, M., and Druker, B. Hematologic and cytogenetic responses to imatinib mesylate in chronic myelogenous leukemia N Engl J Med, 346: 645-652., 2002.
25. Blanke, C. D., Eisenberg, B. L., and Heinrich, M. C. Gastrointestinal stromal tumors. Curr Treat Options Oncol, 2: 485-491., 2001.
26. van Oosterom, A. T., Judson, I., Verweij, J., Stroobants, S., Donato di Paola, E., Dimitijevic, S., Martens, M., Webb, A., Sciot, R., Van Glabbeke, M., Silberman, S., and Nielsen, O. S. Safety and efficacy of imatinib (STI571) in metastatic gastrointestinal stromal tumours: a phase I study. Lancet, 358: 1421-1423., 2001.

1. A method of diagnosing CML or a predisposition to developing CML in a subject, comprising determining a level of expression of a CML-associated gene in a patient derived biological sample, wherein an increase or decrease of said level compared to a normal control level of said gene indicates that said subject suffers from or is at risk of developing CML.

2. The method of claim 1, wherein said CML-associated gene is selected from the group consisting of CML 1-190, wherein an increase in said level compared to a normal control level indicates said subject suffers from or is at risk of developing CML.

3. The method of claim 1, wherein said increase is at least 10% greater than said normal control level.

4. The method of claim 1, wherein said CML-associated gene is selected from the group consisting of CML 191-296, wherein a decrease in said level compared to a normal control level indicates said subject suffers from or is at risk of developing CML.

5. The method of claim 4, wherein said decrease is at least 10% lower than said normal control level.

6. The method of claim 1, wherein said method further comprises determining said level of expression of a plurality of CML-associated genes.

7. The method of claim 1, wherein the expression level is determined by any one method select from group consisting of:
(a) detecting the mRNA of the CML-associated genes,
(b) detecting the protein encoded by the CML-associated genes, and
(c) detecting the biological activity of the protein encoded by the CML-associated genes,

8. The method of claim 1, wherein said level of expression is determined by detecting hybridization of a CML-associated gene probe to a gene transcript of said patient-derived biological sample.

9. The method of claim 1, wherein said hybridization step is carried out on a DNA array

10. The method of claim 1, wherein said biological sample comprises a mononuclear cell.

11. The method of claim 1, wherein said biological sample comprises a myeloid cell.

12. The method of claim 8, wherein said biological sample comprises a lymphoid cell.

13. A CML reference expression profile, comprising a pattern of gene expression of two or more genes selected from the group consisting of CML 1-296.

14. A CML reference expression profile, comprising a pattern of gene expression of two or more genes selected from the group consisting of CML 1-190.

15. A CML reference expression profile, comprising a pattern of gene expression of two or more genes selected from the group consisting of CML 191-296.

16. A method of screening for a compound for treating or preventing CML, said method comprising the steps of:
a) contacting a test compound with a polypeptide encoded by CML 1-296;
b) detecting the binding activity between the polypeptide and the test compound; and
c) selecting a compound that binds to the polypeptide.

17. A method of screening for a compound for treating or preventing CML, said method comprising the steps of:
a) contacting a candidate compound with a cell expressing one or more marker genes, wherein the one or more marker genes is selected from the group consisting of CML 1-296; and
b) selecting a compound that reduces the expression level of one or more marker genes selected from the group consisting of CML 1-190, or elevates the expression level of one or more marker genes selected from the group consisting of CML 191-296.

18. A method of screening for a compound for treating or preventing CML, said method comprising the steps of:
a) contacting a test compound with a polypeptide encoded by selected from the group consisting of CML 1-296;
b) detecting the biological activity of the polypeptide of step (a); and
c) selecting a compound that suppresses the biological activity of the polypeptide encoded by CML 1-190 in comparison with the biological activity detected in the absence of the test compound, or enhances the biological activity of the polypeptide encoded by CML 191-296 in comparison with the biological activity detected in the absence of the test compound.

19. The method of claim 17, wherein said test cell comprises a cell obtained from peripheral blood of CML patient.

20. A method of screening for compound for treating or preventing CML, said method comprising the steps of:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of CML 1-296
b) measuring the activity of said reporter gene; and
c) selecting a compound that reduces the expression level of said reporter gene when said marker gene is an up-regulated marker gene selected from the group consisting of CML 1-190 or that enhances the expression level of said reporter gene when said marker gene is a down-regulated marker gene selected from the group consisting of CML 191-296, as compared to a control.

21. A kit comprising a detection reagent which binds to two or more nucleic acid sequences selected from the group consisting of CML 1-296.

22. An array comprising a nucleic acid which binds to two or more nucleic acid sequences selected from the group consisting of CML 1-296.

23. A method of treating or preventing CML in a subject comprising administering to said subject an antisense composition, said composition comprising a nucleotide sequence complementary to a coding sequence selected from the group consisting of CML 1-190.

24. A method of treating or preventing CML in a subject comprising administering to said subject a siRNA composition, wherein said composition reduces the expression of a nucleic acid sequence selected from the group consisting of CML 1-190.

25. A method for treating or preventing CML in a subject comprising the step of administering to said subject a pharmaceutically effective amount of an antibody or fragment thereof that binds to a protein encoded by any one gene selected from the group consisting of CML 1-190.

26. A method of treating or preventing CML in a subject comprising administering to said subject a vaccine comprising a polypeptide encoded by a nucleic acid selected from the group consisting of CML 1-190 or an immunologically active fragment of said polypeptide, or a polynucleotide encoding the polypeptide.

27. A method of treating or preventing CML in a subject comprising administering to said subject a compound that increases the expression or activity of CML 191-296.

28. A method for treating or preventing CML in a subject, said method comprising the step of administering a compound that is obtained by the method according to any one of claims 16-20.

29. A method of treating or preventing CML in a subject comprising administering to said subject a pharmaceutically effective amount of polynucleotide select from group consisting of CML 191-296, or polypeptide encoded by thereof.

30. A composition for treating or preventing CML, said composition comprising a pharmaceutically effective amount of an antisense polynucleotide or small interfering RNA against a polynucleotide select from group consisting of CML 1-190.

31. A composition for treating or preventing CML, said composition comprising a pharmaceutically effective amount of an antibody or fragment thereof that binds to a protein encoded by any one gene selected from the group consisting of CML 1-190.

32. A composition for treating or preventing CML, said composition comprising a pharmaceutically effective amount of the compound selected by the method of any one of claims 16-20 as an active ingredient, and a pharmaceutically acceptable carrier.
